SEQUENCE LISTING (45 sequences)

SEQ ID NO: 1 - protein, 121 aa, synthetic construct
QVQLVQSGAEVKKPGASVKVSCKASGYTFSIYTIHWVRQAPGQGLEWMGGINPYNGNTKYAQKFQGRVTMTRDTSISTAYMELSRLRSDDTAVYYCAKAPAVFWWTGLDYWGQGTLVTVSS

SEQ ID NO: 2 - protein, 450 aa, synthetic construct
QVQLVQSGAEVKKPGASVKVSCKASGYTFSIYTIHWVRQAPGQGLEWMGGINPYNGNTKYAQKFQGRVTMTRDTSISTAYMELSRLRSDDTAVYYCAKAPAVFWWTGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 8 aa, synthetic construct
GYTFSIYT

SEQ ID NO: 4 - protein, 8 aa, synthetic construct
INPYNGNT

SEQ ID NO: 5 - protein, 14 aa, synthetic construct
AKAPAVFWWTGLDY

SEQ ID NO: 6-10 - skipped (5 entries)

SEQ ID NO: 11 - protein, 123 aa, synthetic construct
EVQLVESGGGLVKPGGSLRLSCAASGFTFSSYGMHWVRQAPGKGLEWVAGISADSGDKNYADSVKGRFTISRDNAKNSLYLQMNSLRAEDTAVYYCAKEFISWIYTFDYLDYWGQGTLVTVSS

SEQ ID NO: 12 - protein, 452 aa, synthetic construct
EVQLVESGGGLVKPGGSLRLSCAASGFTFSSYGMHWVRQAPGKGLEWVAGISADSGDKNYADSVKGRFTISRDNAKNSLYLQMNSLRAEDTAVYYCAKEFISWIYTFDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 13 - protein, 8 aa, synthetic construct
GFTFSSYG

SEQ ID NO: 14 - protein, 8 aa, synthetic construct
ISADSGDK

SEQ ID NO: 15 - protein, 16 aa, synthetic construct
AKEFISWIYTFDYLDY

SEQ ID NO: 16-20 - skipped (5 entries)

SEQ ID NO: 21 - protein, 116 aa, synthetic construct
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTV

SEQ ID NO: 22 - protein, 223 aa, synthetic construct
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 23 - protein, 9 aa, synthetic construct
NDHDIGVYS

SEQ ID NO: 24 - protein, 7 aa, synthetic construct
YFSQSDK

SEQ ID NO: 25 - protein, 10 aa, synthetic construct
AMGARSSVTH

SEQ ID NO: 26 - protein, 121 aa, synthetic construct
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR

SEQ ID NO: 27 - protein, 165 aa, synthetic construct
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 28 - protein, 202 aa, synthetic construct
  VARIANT 12: note = Any amino acid
  VARIANT 14: note = Any amino acid
  VARIANT 17: note = Any amino acid
  VARIANT 18: note = Any amino acid
  VARIANT 19: note = Any amino acid
  VARIANT 21: note = Any amino acid
  VARIANT 31: note = Any amino acid
  VARIANT 36: note = Any amino acid
  VARIANT 45: note = Any amino acid
  VARIANT 48: note = Any amino acid
  VARIANT 50: note = Any amino acid
  VARIANT 54: note = Any amino acid
  VARIANT 55: note = Any amino acid
  VARIANT 64: note = Any amino acid
  VARIANT 91: note = Any amino acid
  VARIANT 94: note = Any amino acid
  VARIANT 95: note = Any amino acid
  VARIANT 96: note = Any amino acid
  VARIANT 97: note = Any amino acid
  VARIANT 99: note = Any amino acid
  VARIANT 106: note = Any amino acid
  VARIANT 109: note = Any amino acid
  VARIANT 110: note = Any amino acid
  VARIANT 117: note = Any amino acid
  VARIANT 119: note = Any amino acid
  VARIANT 120: note = Any amino acid
  VARIANT 122: note = Any amino acid
  VARIANT 127: note = Any amino acid
  VARIANT 130: note = Any amino acid
  VARIANT 132: note = Any amino acid
  VARIANT 135: note = Any amino acid
  VARIANT 137: note = Any amino acid
  VARIANT 138: note = Any amino acid
  VARIANT 139: note = Any amino acid
  VARIANT 140: note = Any amino acid
  VARIANT 144: note = Any amino acid
  VARIANT 147: note = Any amino acid
  VARIANT 149: note = Any amino acid
  VARIANT 151: note = Any amino acid
  VARIANT 152: note = Any amino acid
  VARIANT 154: note = Any amino acid
  VARIANT 156: note = Any amino acid
  VARIANT 159: note = Any amino acid
  VARIANT 161: note = Any amino acid
  VARIANT 168: note = Any amino acid
  VARIANT 173: note = Any amino acid
  VARIANT 179: note = Any amino acid
  VARIANT 180: note = Any amino acid
  VARIANT 181: note = Any amino acid
  VARIANT 182: note = Any amino acid
  VARIANT 183: note = Any amino acid
  VARIANT 184: note = Any amino acid
  VARIANT 185: note = Any amino acid
  VARIANT 186: note = Any amino acid
  VARIANT 187: note = Any amino acid
  VARIANT 188: note = Any amino acid
  VARIANT 189: note = Any amino acid
  VARIANT 190: note = Any amino acid
  VARIANT 191: note = Any amino acid
  VARIANT 192: note = Any amino acid
  VARIANT 193: note = Any amino acid
  VARIANT 194: note = Any amino acid
  VARIANT 195: note = Any amino acid
  VARIANT 196: note = Any amino acid
MLPAVPPQQWPXSXGRXXXHXEVVKFIDVWXRSYCXPREVLVDVXSEXPXEVEXXFKPSCVSVXRCGGCCNDEGLECVPTETSNVTMQLLXIRXXXXPXLVEMSFXQHXXCECRPKXKXXAXKPDRXADXCXRRXPXXXXPDPXTCXCXCXXTXSXRCXAXGLELNEXTCRCXKLRRGXXXXXXXXXXXXXXXXXXDAVPRR

SEQ ID NO: 29 - protein, 164 aa, synthetic construct
APMAEGGGQNHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 30 - protein, 132 aa, synthetic construct
MLPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 31 - protein, 203 aa, synthetic construct
LPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 32 - protein, 186 aa, synthetic construct
PVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 33 - protein, 167 aa, synthetic construct
PVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 34 - protein, 116 aa, synthetic construct
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 35 - protein, 117 aa, synthetic construct
FAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRR

SEQ ID NO: 36 - protein, 165 aa, synthetic construct
APMAEGGDQKPHEVVKFMDVYQRSYCRPIETLVDIFQEYPDEIEYIFKPSCVPLMRCGGCCNDEGLECVPTEEFNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 37 - protein, 165 aa, Oryctolagus sp.
APMAEEGDNKPHEVVKFMEVYRRSYCQPIETLVDIFQEYPDEIEYIFKPSCVPLVRCGGCCNDESLECVPTEEFNVTMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 38 - protein, 163 aa, Canis lupus
PMAGGEHKPHEVVKFMDVYQRSYCRPIETLVDIFQEYPDEIEYIFKPSCVPLMRCGGCCNDEGLECVPTEEFNITMQIMRIKPHQGQHIGEMSFLQHSKCECRPKKDRARQENPCGPCSERRKHLFVQDPQTCKCSCKNTDSRCKARQLELNERTCRCDKPRR

SEQ ID NO: 39 - protein, 164 aa, Sus scrofa
APMAEGDQKPHEVVKFMDVYQRSYCRPIETLVDIFQEYPDEIEYIFKPSCVPLMRCGGCCNDEGLECVPTEEFNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 40 - protein, 496 aa, synthetic construct
  VARIANT 31: note = Any amino acid
  VARIANT 32: note = Any amino acid
  VARIANT 56: note = Any amino acid
  VARIANT 257: note = Any amino acid
  VARIANT 295: note = Any amino acid
  VARIANT 309: note = Any amino acid
  VARIANT 363: note = Any amino acid
  VARIANT 376: note = Any amino acid
  VARIANT 397: note = Any amino acid
MWQIVFFTLSCDLVLAAAYNNFRKSMDSIGXXQYQVQHGSCSYTFLLPETDNCRSXSSPYVSNAVQRDAPLDYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLDMEDKHIVQLQSIKEEKDQLQVLVSKQNSIIEELEKQLVTATVNNSVLQKQQHDLMETVHXLLTMISTSNSAKDSFVAKEEQIIFRDCAEVFKSGLTTXGIYTLTFPNSTEEXKAYCDMETGGGGWTVIQRREDGSVDFQRTWKEYKVGFGNPSGEHWLGNEFVSQXTNQQRYVLKIHLXDWEGNEAYSLYEHFYLSSEEXNYRIHLKGLTGTAGKISSISQPGNDFSTKDADNDKCICKCSQMLTGGWWFDACGPSNLNGMYYPQRQNTNKFNGIKWYYWKGSGYSLKATTMMIRPADF

SEQ ID NO: 41 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 42 - protein, 496 aa, Oryctolagus sp.
MWRIVFFSLSCDLVLAAAYSNFRKSMDSVGRKQYQVQHGSCSYTFLLPETDNCRSSSSPYVSNAVQRDAPLDYDDSVHRLQVLESIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLDMEDKHIVQLQSIKEEKDQLQVLVSKQNSIIEELERQLVTATVNNSALQKQQHDLTETVHSLLTMIATSSSAKNSFAAKEEQITFRDCADVFRSGLTTSGIYTLTFPNSTEETKAYCDMETGGGGWTTIQRREDGSVDFQRTWKEYKVGFGNPSGEHWLGNEFVSQLTNQQRYVLKIHLRDWEGNEAYSLYEHFYLSSEEFNYRIHLKGLTGTAGKISSISQPGNDFSTKDADNDKCICKCSQMLTGGWWFDACGPSNLNGMYYPQRQNTNKFNGIKWYYWKGSGYSLKATTMMIRPADF

SEQ ID NO: 43 - protein, 495 aa, Canis lupus
MWQIVFFTLSCDLVRAAAYNNFRRSMDSIGRRQYQVQHGSCSYTFLLPETDNCRSPGSYVPNAVQRDAPLDYDDSVQRLQVLENIMENNTQWLIKLENYIQDNMKKEMVEMQQNAVQNQTAVMIEIGTNLLNQTAEQTRKLTDVEAQVLNQTTRLELQLLEHSLSTNKLEKQILDQTSEINKLQDKNSFLEKKVLDMEDKHIVQLRSIKEEKDQLQVLVSKQNSIIEELEKQLVTATVNNSVLQKQQHDLMETVHSLLTMISPSKSPKDTFVAKEEQIIYRDCAEVFKSGLTTNGIYTLTFPNSTEEIKAYCDMETSGGGWTVIQRREDGSVDFQRTWKEYKVGFGNPSGEHWLGNEFVFQVTNQQPYVLKIHLKDWEGNEAYSLYEHFYLSGEELNYRIHLKGLTGTAGKISSISQPGNDFSTKDADNDKCICKCSQMLTGGWWFDACGPSNLNGMYYPQRQNTNKFNGIKWYYWKGSGYSLKGTTMMIRPADF

SEQ ID NO: 44 - protein, 496 aa, Sus scrofa
MWQLVFFALSCDLVLAAAYNNFRKSMDSTGKRQYQVQHGPCSYTFLLPETDNCRSPSSSYVSNAVQRDAPLDYDDSVRRLQVLENIMENNTQWLMKLESYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLDMEDKHIVQLQSIKEEKDQLQVLVSKQNSIIEELEKQLVTATVNNSVLQKQQHDLMETVHNLLTMISTSNSAKHSLVAKEEQIIFRDCAEAFKSGLTTSGTYTLTFPNSTEETKAYCDMETGGGGWTVIQRREDGSVDFQRTWKEYKMGFGSPSGEHWLGNEFVSQVTNQKRYVLKIHLRDWEGNEAYSLYEHFYLSSEEFNYRIHLKGLTGTAGKISSISQPGNDFSTKDADNDKCICKCSQMLTGGWWFDACGPSNLNGMYYPQRQNTNKFNGIKWYYWKGSGYSLKATTMMIRPADF

SEQ ID NO: 45 - protein, 165 aa, Homo sapiens; identical to SEQ ID NO: 27

(19) United States
(12) Patent Application Publication
(10) Pub. No.: US 2025/0250330 A1
(43) Pub. Date: Aug. 7, 2025

(54) ANG-2/VEGF ANTIBODIES AND USES THEREOF

(71) Applicant: RevOpsis Therapeutics, Inc., Springfield, IL (US)

(72) Inventors: Ramanath BHANDARI, Springfield, IL (US); Li XU, Springfield, IL (US); Ramesh BHATT, Springfield, IL (US); Ram Prashanth PALANKI, Springfield, IL (US)

(21) Appl. No.: 19/013,495

(22) Filed: Jan. 8, 2025

Related U.S. Application Data
(63) Continuation of application No. PCT/US2023/069962, filed on Jul. 11, 2023.
(60) Provisional application No. 63/388,538, filed on Jul. 12, 2022.

(51) Int. Cl. C07K 16/22

(52) U.S. Cl. CPC C07K 16/22; C07K 2317/31; C07K 2317/565; C07K 2317/92

(57) ABSTRACT

Described herein are antibodies that bind VEGF_A or ANG-2, or bispecific antibodies that bind VEGF_A and ANG-2. Antibodies of this disclosure are useful for treating diseases of the eye including macular degeneration.

### CROSS-REFERENCE

This application is a continuation of International Application No. PCT/US2023/069962, filed Jul. 11, 2023, which claims the benefit of U.S. Provisional Application No. 63/388,538, filed Jul. 12, 2022, which application is incorporated herein by reference.

### BACKGROUND

Diseases of the eye including macular degeneration and retinal vein occlusions afflict thousands of individuals each year and can result in permanently reduced visual acuity or blindness. The proteins vascular endothelial growth factor (VEGF) and angiopoietin-2 (ANG2) play a role in the development of these diseases thus serve as a potential therapeutic target.

### SEQUENCE LISTING

The application contains a Sequence Listing which has been submitted in XML format via Patent Center and is hereby incorporated by reference in its entirety. Said XML copy, created on Aug. 29, 2023, is named “63209-701_601_SL.xml” and 65k bytes in size.

### SUMMARY

Described herein are antibodies and bispecific antibodies that that bind to VEGF and/or ANG-2. Also described are methods of treating diseases of the eye by administering the antibodies and bispecific antibodies to individuals in need thereof. Such diseases include macular degeneration, retinal vein occlusions, branch vein occlusions, and central vein occlusions. The bispecific antibodies described herein possess advantages over previously described bispecific antibodies in that the binding moieties that bind to VEGF and ANG-2 both bind with high-affinity to their respective antigens. Further, the surrogate light chains, which are used as the common light chin are of human germline origin, and are, thus, less immunogenic than other common light chains allowing for a safer molecule that is less likely to be subject to anti-therapeutic antibody responses. Low anti-therapeutic antibody immune responses are desirable as it allows the therapeutic antibody to maintain efficacy over repeat administrations.

As described herein, in one aspect, is a vascular endothelial growth factor (VEGF) binding antibody or antigen binding fragment thereof comprising: (a) a heavy chain complementarity determining region 1 (HCDR1) comprising the amino acid sequence set forth in SEQ ID NO: 3 (GYTFSIYT); (b) a heavy chain complementarity determining region 2 (HCDR2) comprising the amino acid sequence set forth in SEQ ID NO: 4 (INPYNGNT); and (c) a heavy chain complementarity determining region 3 (HCDR3) comprising the amino acid sequence set forth in SEQ ID NO: 5 (AKAPAVFWWTGLDY); wherein the antibody binds to VEGF. In certain embodiments, the antibody or antigen binding fragment thereof comprises a heavy chain variable region comprising an amino acid sequence comprising at least 85%, 90%, 95%, 96%, 97%, 98%, 99%, or 100% identity to the amino acid sequence set forth in SEQ ID NO: 1. In certain embodiments, the antibody or antigen binding fragment thereof comprises a heavy chain constant region comprising an amino acid sequence comprising at least 85%, 90%, 95%, 96%, 97%, 98%, 99%, or 100% identity to the amino acid sequence set forth in SEQ ID NO: 2. In certain embodiments, the antibody or antigen binding fragment thereof comprises a surrogate light chain. In certain embodiments, the surrogate light chain comprises: (a) a light chain complementarity determining region 1 (LCDR1) comprising the amino acid sequence set forth in SEQ ID NO: 23 (NDHDIGVYS); (b) a light chain complementarity determining region 2 (LCDR2) comprising the amino acid sequence set forth in SEQ ID NO: 24 (YFSQSDK); and (c) a light chain complementarity determining region 3 (LCDR3) comprising the amino acid sequence set forth in SEQ ID NO: 25 (AMGARSSVTH). In certain embodiments, the antibody or antigen binding fragment thereof comprises a light chain variable region comprising an amino acid sequence comprising at least 85%, 90%, 95%, 96%, 97%, 98%, 99%, or 100% identity to the amino acid sequence set forth in SEQ ID NO: 21. In certain embodiments, the antibody or antigen binding fragment thereof comprises a light chain constant region comprising an amino acid sequence comprising at least 85%, 90%, 95%, 96%, 97%, 98%, 99%, or 100% identity to the amino acid sequence set forth in SEQ ID NO: 22. In certain embodiments, the antibody binds to a peptide comprising a platelet-derived growth factor (PDGF) domain of VEGF-A. In certain embodiments, the PDGF domain of VEGF_A comprises a fragment of VEGF_A ranging from 50th to 132nd amino acids. In certain embodiments, the antibody binds to a peptide comprising a VEGF-C heparin domain of VEGF-A. In certain embodiments, the VEGF_C heparin domain of VEGF_A comprises a fragment of VEGF-A ranging from 143rd to 191st amino acids. In certain embodiments, the antibody binds to VEGF-A or a fragment thereof. In certain embodiments, the antibody binds to VEGF_A or a fragment thereof with an EC50 of about 60 picomolar or less. In certain embodiments, the antibody inhibits an interaction of VEGF-A or a fragment thereof and a VEGF receptor. In certain embodiments, the antibody inhibits the interaction of VEGF_A or a fragment thereof and the VEGF receptor with an IC50 of about 2000 picomolar or less. In certain embodiments, the antibody inhibits the interaction of VEGF_A or a fragment thereof and the VEGF receptor at least 3-fold stronger than Faricimab inhibits the interaction. In certain embodiments, the VEGF receptor is a VEGF receptor 2/kinase insert domain receptor (KDR). In certain embodiments, the antibody or antigen binding fragment thereof is a Fab, F(ab)2, a single-domain antibody, or a single chain variable fragment (scFv). In certain embodiments, the antibody or antigen binding fragment thereof is an IgG antibody. In certain embodiments, the antibody or antigen binding fragment thereof is human, chimeric, or humanized. In certain embodiments, the antibody or antigen binding fragment thereof is humanized. Also described is a pharmaceutical composition comprising the antibody or antigen binding fragment thereof and a pharmaceutically acceptable carrier, excipient, or diluent. In certain embodiments, the pharmaceutical composition is formulated for intravenous administration. In certain embodiments, the pharmaceutical composition is formulated for intravitreal administration. Also described herein is a nucleic acid encoding the antibody. In certain embodiments, the nucleic acid is an expression vector. In certain embodiments, the expression vector is a lentiviral vector, an adenoviral vector, an adeno-associated viral vector, or a plasmid vector. In certain embodiments, the expression vector is formulated for intravenous administration. In certain embodiments, the expression vector is formulated for intravitreal administration. Also described herein is a cell comprising the nucleic acid or the expression vector. In certain embodiments, the cell is a eukaryotic cell suitable for antibody production. In certain embodiments, the bispecific antibody is used in a method of treating a disease of the eye in an individual, the method comprising administering to the individual the antibody, the nucleic acid or plurality of nucleic acids encoding the antibody, or pharmaceutical composition comprising the antibody, thereby treating the disease of the eye. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

As described herein, in one aspect, is a angiopoietin 2 (ANG-2) binding antibody or antigen binding fragment thereof comprising: (a) a heavy chain complementarity determining region 1 (HCDR1) comprising the amino acid sequence set forth in SEQ ID NO: 13 (GFTFSSYG); (b) a heavy chain complementarity determining region 2 (HCDR2) comprising the amino acid sequence set forth in SEQ ID NO: 14 (ISADSGDK); and (c) a heavy chain complementarity determining region 3 (HCDR3) comprising the amino acid sequence set forth in SEQ ID NO: 15 (AKEFISWIYTFDYLDY), wherein the antibody binds to ANG-2. In certain embodiments, the antibody or antigen binding fragment thereof comprises an amino acid sequence comprising at least 85%, 90%, 95%, 96%, 97%, 98%, 99%, or 100% identity to the amino acid sequence set forth in SEQ ID NO: 11. In certain embodiments, the antibody or antigen binding fragment thereof comprises a heavy chain constant region comprising an amino acid sequence comprising at least 85%, 90%, 95%, 96%, 97%, 98%, 99%, or 100% identity to the amino acid sequence set forth in SEQ ID NO: 12. In certain embodiments, the antibody or antigen binding fragment thereof comprises a surrogate light chain. In certain embodiments, the surrogate light chain comprises: (a) a light chain complementarity determining region 1 (LCDR1) comprising the amino acid sequence set forth in SEQ ID NO: 23 (NDHDIGVYS); (b) a light chain complementarity determining region 2 (LCDR2) comprising the amino acid sequence set forth in SEQ ID NO: 24 (YFSQSDK); and (c) a light chain complementarity determining region 3 (LCDR3) comprising the amino acid sequence set forth in SEQ ID NO: 25 (AMGARSSVTH). In certain embodiments, the antibody or antigen binding fragment thereof comprises a light chain variable region comprising an amino acid sequence comprising at least 85%, 90%, 95%, 96%, 97%, 98%, 99%, or 100% identity to the amino acid sequence set forth in SEQ ID NO: 21. In certain embodiments, the antibody or antigen binding fragment thereof comprises a light chain constant region comprising an amino acid sequence comprising at least 85%, 90%, 95%, 96%, 97%, 98%, 99%, or 100% identity to the amino acid sequence set forth in SEQ ID NO: 22. In certain embodiments, the antibody binds ANG-2. In certain embodiments, the antibody binds ANG-2 with an EC50 of about 55 picomolar or less. In certain embodiments, the antibody binds ANG-2 at least 50-fold stronger than Faricimab binds to ANG-2. In certain embodiments, the antibody inhibits an interaction of ANG-2 and an ANG-2 receptor. In certain embodiments, the antibody inhibits the interaction of ANG-2 and the ANG-2 receptor with an IC50 of about 800 picomolar or less. In certain embodiments, the antibody inhibits the interaction of ANG-2 and the ANG-2 receptor at least 15-fold stronger than Faricimab. In certain embodiments, the ANG-2 receptor is a tyrosine kinase with immunoglobulin-like and EGF-like domains 2 receptor (Tie-2 receptor). In certain embodiments, the antibody or antigen binding fragment thereof is a Fab, F(ab)2, a single-domain antibody, or a single chain variable fragment (scFv). In certain embodiments, the antibody or antigen binding fragment thereof is an IgG antibody. In certain embodiments, the antibody or antigen binding fragment thereof is human, chimeric, or humanized. In certain embodiments, the antibody or antigen binding fragment thereof is humanized. Also described is a pharmaceutical composition comprising the antibody or antigen binding fragment thereof and a pharmaceutically acceptable carrier, excipient, or diluent. In certain embodiments, the pharmaceutical composition is formulated for intravenous administration. In certain embodiments, the pharmaceutical composition is formulated for intravitreal administration. Also described herein is a nucleic acid encoding the antibody. In certain embodiments, the nucleic acid is an expression vector. In certain embodiments, the expression vector is a lentiviral vector, an adenoviral vector, an adeno-associated viral vector, or a plasmid vector. In certain embodiments, the expression vector is formulated for intravenous administration. In certain embodiments, the expression vector is formulated for intravitreal administration. Also described herein is a cell comprising the nucleic acid or the expression vector. In certain embodiments, the cell is a eukaryotic cell suitable for antibody production. In certain embodiments, the antibody is used in a method of treating a disease of the eye in an individual, the method comprising administering to the individual the antibody, the nucleic acid or plurality of nucleic acids encoding the antibody, or pharmaceutical composition comprising the antibody, thereby treating the disease of the eye. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

Described herein in another aspect is a bispecific antibody comprising: a vascular endothelial growth factor (VEGF) binding moiety comprising: (a) a heavy chain complementarity determining region 1 (HCDR1) comprising the amino acid sequence set forth in SEQ ID NO: 3 (GYTFSIYT); (b) a heavy chain complementarity determining region 2 (HCDR2) comprising the amino acid sequence set forth in SEQ ID NO: 4 (INPYNGNT); and (c) a heavy chain complementarity determining region 3 (HCDR3) comprising the amino acid sequence set forth in SEQ ID NO: 5 (AKAPAVFWWTGLDY); and an angiopoietin 2 (ANG-2) binding moiety comprising: (a) a heavy chain complementarity determining region 1 (HCDR1) comprising the amino acid sequence set forth in SEQ ID NO: 13 (GFTFSSYG); (b) a heavy chain complementarity determining region 2 (HCDR2) comprising the amino acid sequence set forth in SEQ ID NO: 14 (ISADSGDK); and (c) a heavy chain complementarity determining region 3 (HCDR3) comprising the amino acid sequence set forth in SEQ ID NO: 15 (AKEFISWIYTFDYLDY); wherein the bispecific antibody binds to VEGF and ANG-2. In certain embodiments, the VEGF binding moiety comprises a heavy chain variable region comprising an amino acid sequence at least 85%, 90%, 95%, 96%, 97%, 98%, 99%, or 100% identity to the amino acid sequence set forth in SEQ ID NO: 1. In certain embodiments, the VEGF binding moiety further comprises a heavy chain constant region. In certain embodiments, the heavy chain constant region comprises an engineered protuberance or an engineered cavity such that homodimerization of the VEGF binding moiety is inhibited. In certain embodiments, the engineered protuberance comprises a T366W substitution according to EU numbering. In certain embodiments, the engineered cavity comprises a T366S/L368A/Y407V substitution according to EU numbering. In certain embodiments, the heavy chain constant region comprises one or more substitutions to the Fc region that reduces antibody effector function. In certain embodiments, the one or more substitutions to the Fc region that reduces antibody effector function comprise L234A and/or L235A according to EU numbering. In certain embodiments, the heavy chain constant region comprises one or more substitutions of a cysteine residue. In certain embodiments, the one or more substitutions of a cysteine residue is at serine 354 by EU numbering. In certain embodiments, the VEGF binding moiety comprises a heavy chain constant region comprising an amino acid sequence comprising at least 85%, 90%, 95%, 96%, 97%, 98%, 99%, or 100% identity to the amino acid sequence set forth in SEQ ID NO: 2. In certain embodiments, the ANG-2 binding moiety comprises a heavy chain variable region comprising an amino acid sequence comprising at least 85%, 90%, 95%, 96%, 97%, 98%, 99%, or 100% identity to the amino acid sequence set forth in SEQ ID NO: 11. In certain embodiments, the ANG-2 binding moiety further comprises a heavy chain constant region. In certain embodiments, the heavy chain constant region comprises an engineered protuberance or an engineered cavity such that homodimerization of the ANG-2 binding moiety is inhibited. In certain embodiments, the engineered protuberance comprises a T366W substitution according to EU numbering. In certain embodiments, the engineered cavity comprises a T366S/L368A/Y407V substitution according to EU numbering. In certain embodiments, the heavy chain constant region comprises one or more substitutions to the Fc region that reduces antibody effector function. In certain embodiments, the one or more substitutions to the Fc region that reduces antibody effector function comprise L234A and/or L235A according to EU numbering. In certain embodiments, the heavy chain constant region comprises one or more substitutions of a cysteine residue. In certain embodiments, the one or more substitutions of a cysteine residue is at serine 354 by EU numbering. In certain embodiments, the bispecific antibody binds to a peptide comprising a platelet-derived growth factor (PDGF) domain of VEGF-A. In certain embodiments, the PDGF domain of VEGF_A comprises a fragment of VEGF_A ranging from 50th to 132nd amino acids. In certain embodiments, the bispecific antibody binds to a peptide comprising a VEGF-C heparin domain of VEGF-A. In certain embodiments, the VEGF_C heparin domain of VEGF_A comprises a fragment of VEGF-A ranging from 143rd to 191st amino acids. In certain embodiments, the bispecific antibody binds to VEGF-A or a fragment thereof. In certain embodiments, the bispecific antibody binds to VEGF_A or a fragment thereof with an EC50 of about 60 picomolar or less. In certain embodiments, the bispecific antibody inhibits an interaction of VEGF-A or a fragment thereof and a VEGF receptor. In certain embodiments, the bispecific antibody inhibits the interaction of VEGF_A or a fragment thereof and the VEGF receptor with an IC50 of about 2000 picomolar or less. In certain embodiments, the bispecific antibody inhibits the interaction of VEGF_A or a fragment thereof and the VEGF receptor at least 3-fold stronger than Faricimab inhibits the interaction. In certain embodiments, the VEGF receptor is a VEGF receptor 2/kinase insert domain receptor (KDR). In certain embodiments, the ANG-2 binding moiety comprises a heavy chain constant region comprising an amino acid sequence comprising at least 85%, 90%, 95%, 96%, 97%, 98%, 99%, or 100% identity to the amino acid sequence set forth in SEQ ID NO: 12. In certain embodiments, the bispecific antibody comprises a common light chain. In certain embodiments, the common light chain is a surrogate light chain. In certain embodiments, the surrogate light chain comprises: (a) a light chain complementarity determining region 1 (LCDR1) comprising the amino acid sequence set forth in SEQ ID NO: 23 (NDHDIGVYS); (b) a light chain complementarity determining region 2 (LCDR2) comprising the amino acid sequence set forth in SEQ ID NO: 24 (YFSQSDK); and (c) a light chain complementarity determining region 3 (LCDR3) comprising the amino acid sequence set forth in SEQ ID NO: 25 (AMGARSSVTH). In certain embodiments, the surrogate light chain comprises a light chain variable region comprising an amino acid sequence comprising at least 85%, 90%, 95%, 96%, 97%, 98%, 99%, or 100% identity to the amino acid sequence set forth in SEQ ID NO: 21. In certain embodiments, the surrogate light chain comprises a light chain constant region comprising an amino acid sequence comprising at least 85%, 90%, 95%, 96%, 97%, 98%, 99%, or 100% identity to the amino acid sequence set forth in SEQ ID NO: 22. In certain embodiments, the bispecific antibody comprises: (a) a first heavy chain variable region comprising an amino acid sequence comprising at least 85%, 90%, 95%, 96%, 97%, 98%, 99%, 100% identity to the amino acid sequence set forth in SEQ ID NO: 1; (b) a second heavy chain variable region comprising an amino acid sequence comprising at least 85%, 90%, 95%, 96%, 97%, 98%, 99%, 100% identity to the amino acid sequence set forth in SEQ ID NO: 11; and (c) a common light chain variable region, wherein the common light chain variable region comprises a light chain variable region comprising an amino acid sequence comprising at least 85%, 90%, 95%, 96%, 97%, 98%, 99%, 100% identity to the amino acid sequence set forth in SEQ ID NO: 21, wherein the bispecific antibody binds to VEGF and ANG-2. In certain embodiments, the bispecific antibody binds ANG-2. In certain embodiments, the bispecific antibody binds ANG-2 with an EC50 of about 55 picomolar or less. In certain embodiments, the bispecific antibody binds ANG-2 at least 50-fold stronger than Faricimab binds to ANG-2. In certain embodiments, the bispecific antibody inhibits an interaction of ANG-2 and an ANG-2 receptor. In certain embodiments, the bispecific antibody inhibits the interaction of ANG-2 and the ANG-2 receptor with an IC50 of about 800 picomolar or less. In certain embodiments, the bispecific antibody inhibits the interaction of ANG-2 and the ANG-2 receptor at least 15-fold stronger than Faricimab. In certain embodiments, the ANG-2 receptor is a tyrosine kinase with immunoglobulin-like and EGF-like domains 2 receptor (Tie-2 receptor). In certain embodiments, the bispecific antibody or antigen binding fragment thereof is human, chimeric, or humanized. In certain embodiments, the bispecific antibody or antigen binding fragment thereof is humanized. Also described is a pharmaceutical composition comprising the bispecific antibody or antigen binding fragment thereof and a pharmaceutically acceptable carrier, excipient, or diluent. In certain embodiments, the pharmaceutical composition is formulated for intravenous administration. In certain embodiments, the pharmaceutical composition is formulated for intravitreal administration. Also described herein is a nucleic acid encoding the bispecific antibody. In certain embodiments, the nucleic acid is an expression vector. In certain embodiments, the expression vector is a lentiviral vector, an adenoviral vector, an adeno-associated viral vector, or a plasmid vector. In certain embodiments, the expression vector is formulated for intravenous administration. In certain embodiments, the expression vector is formulated for intravitreal administration. Also described herein is a cell comprising the nucleic acid or the expression vector. In certain embodiments, the cell is a eukaryotic cell suitable for bispecific antibody production. In certain embodiments, the bispecific antibody is used in a method of treating a disease of the eye in an individual, the method comprising administering to the individual the bispecific antibody, the nucleic acid or plurality of nucleic acids encoding the bispecific antibody, or pharmaceutical composition comprising the bispecific antibody, thereby treating the disease of the eye. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

Described herein, in another aspect, is a bispecific antibody comprising: (a) a first heavy chain comprising an amino acid sequence comprising at least 85%, 90%, 95%, 96%, 97%, 98%, 99%, 100% identity to the amino acid sequence set forth in SEQ ID NO: 2; (b) a second heavy chain comprising an amino acid sequence comprising at least 85%, 90%, 95%, 96%, 97%, 98%, 99%, 100% identity to the amino acid sequence set forth in SEQ ID NO: 12; and (c) a common light chain, wherein the common light chain comprises a light chain comprising an amino acid sequence comprising at least 85%, 90%, 95%, 96%, 97%, 98%, 99%, 100% identity to the amino acid set forth in SEQ ID NO: 22, wherein the bispecific antibody binds to VEGF and ANG-2. In certain embodiments, the bispecific antibody binds VEGF with an EC50 of about 60 picomolar or less. In certain embodiments, the bispecific antibody inhibits the interaction of VEGF_A or a fragment thereof and the VEGF receptor with an IC50 of about 2000 picomolar or less. In certain embodiments, the bispecific antibody binds ANG-2 at least 50-fold stronger than Faricimab binds to ANG-2. In certain embodiments, the bispecific antibody inhibits an interaction of ANG-2 and an ANG-2 receptor. In certain embodiments, the bispecific antibody inhibits the interaction of ANG-2 and the ANG-2 receptor with an IC50 of about 800 picomolar or less. In certain embodiments, the bispecific antibody inhibits the interaction of ANG-2 and the ANG-2 receptor at least 15-fold stronger than Faricimab. In certain embodiments, the bispecific antibody inhibits the interaction of VEGF_A or a fragment thereof and the VEGF receptor at least 3-fold stronger than Faricimab inhibits the interaction. In certain embodiments, the bispecific antibody binds ANG-2 with an EC50 of about 55 picomolar or less. In certain embodiments, the bispecific antibody or antigen binding fragment thereof is human, chimeric, or humanized. In certain embodiments, the bispecific antibody or antigen binding fragment thereof is humanized. Also described is a pharmaceutical composition comprising the bispecific antibody or antigen binding fragment thereof and a pharmaceutically acceptable carrier, excipient, or diluent. In certain embodiments, the pharmaceutical composition is formulated for intravenous administration. In certain embodiments, the pharmaceutical composition is formulated for intravitreal administration. Also described herein is a nucleic acid encoding the bispecific antibody. In certain embodiments, the nucleic acid is an expression vector. In certain embodiments, the expression vector is a lentiviral vector, an adenoviral vector, an adeno-associated viral vector, or a plasmid vector. In certain embodiments, the expression vector is formulated for intravenous administration. In certain embodiments, the expression vector is formulated for intravitreal administration. Also described herein is a cell comprising the nucleic acid or the expression vector. In certain embodiments, the cell is a eukaryotic cell suitable for bispecific antibody production. In certain embodiments, the bispecific antibody is used in a method of treating a disease of the eye in an individual, the method comprising administering to the individual the bispecific antibody, the nucleic acid or plurality of nucleic acids encoding the bispecific antibody, or pharmaceutical composition comprising the bispecific antibody, thereby treating the disease of the eye. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

Described herein, in another aspect, is a bispecific antibody comprising: (a) a first heavy chain comprising an amino acid sequence as set forth in SEQ ID NO: 2; (b) a second heavy chain comprising an amino acid sequence as set forth in SEQ ID NO: 12; and (c) a common light chain, wherein the common light chain comprises a light chain comprising an as set forth in SEQ ID NO: 22, wherein the bispecific antibody binds to VEGF and ANG-2. In certain embodiments, the bispecific antibody binds VEGF with an EC50 of about 60 picomolar or less. In certain embodiments, the bispecific antibody inhibits an interaction of VEGF-A or a fragment thereof and a VEGF receptor. In certain embodiments, the bispecific antibody inhibits the interaction of VEGF_A or a fragment thereof and the VEGF receptor with an IC50 of about 2000 picomolar or less. In certain embodiments, the bispecific antibody inhibits the interaction of VEGF_A or a fragment thereof and the VEGF receptor at least 3-fold stronger than Faricimab inhibits the interaction. In certain embodiments, the bispecific antibody binds ANG-2 with an EC50 of about 55 picomolar or less. In certain embodiments, the bispecific antibody binds ANG-2 at least 50-fold stronger than Faricimab binds to ANG-2. In certain embodiments, the bispecific antibody inhibits an interaction of ANG-2 and an ANG-2 receptor. In certain embodiments, the bispecific antibody inhibits the interaction of ANG-2 and the ANG-2 receptor with an IC50 of about 800 picomolar or less. In certain embodiments, the bispecific antibody inhibits the interaction of ANG-2 and the ANG-2 receptor at least 15-fold stronger than Faricimab. In certain embodiments, the bispecific antibody or antigen binding fragment thereof is human, chimeric, or humanized. In certain embodiments, the bispecific antibody or antigen binding fragment thereof is humanized. Also described is a pharmaceutical composition comprising the bispecific antibody or antigen binding fragment thereof and a pharmaceutically acceptable carrier, excipient, or diluent. In certain embodiments, the pharmaceutical composition is formulated for intravenous administration. In certain embodiments, the pharmaceutical composition is formulated for intravitreal administration. Also described herein is a nucleic acid encoding the bispecific antibody. In certain embodiments, the nucleic acid is an expression vector. In certain embodiments, the expression vector is a lentiviral vector, an adenoviral vector, an adeno-associated viral vector, or a plasmid vector. In certain embodiments, the expression vector is formulated for intravenous administration. In certain embodiments, the expression vector is formulated for intravitreal administration. Also described herein is a cell comprising the nucleic acid or the expression vector. In certain embodiments, the cell is a eukaryotic cell suitable for bispecific antibody production. In certain embodiments, the bispecific antibody is used in a method of treating a disease of the eye in an individual, the method comprising administering to the individual the bispecific antibody, the nucleic acid or plurality of nucleic acids encoding the bispecific antibody, or pharmaceutical composition comprising the bispecific antibody, thereby treating the disease of the eye. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIGS. 1A-1B illustrate SDS-PAGE and SE-HPLC analysis of bispecific surrobodies with amino acid sequences corresponding to SEQ ID NO: 2, SEQ ID NO: 12, and SEQ ID NO: 22 produced in CHO cells. FIG. 1A illustrates SDS-PAGE results for reduced and non-reduced samples. Lane 1, MW ladder; lane 2, IgG control (non-reduced); lane 3, Sample 1 (non-reduced); lane 4, Sample 2 (non-reduced); lane 5, Sample 3 (non-reduced); lane 6, IgG control (reduced); lane 7, Sample 1 (reduced); lane 8, Sample 2 (reduced); lane 5, Sample 3 (reduced). FIG. 1B illustrates SE-HPLC results of the 3 samples.

FIGS. 2A-2F illustrate surrobody RO-101's binding to VEGF and its inhibition of the interaction between VEGF and its receptors in comparison with Faricimab. FIG. 2A illustrates sequence alignment between VEGF₁₂₁ and VEGF₁₆₅. FIG. 2B illustrates ELISA-binding results for RO-101's binding to VEGF₁₂₁ or VEGF₁₆₅. FIG. 2C illustrates ELISA-binding results for RO-101 or Faricimab's binding to VEGF₁₂₁. FIG. 2D illustrates ELISA-binding results for RO-101 or Faricimab's binding to VEGF₁₆₅. FIG. 2E illustrates a diagram of an assay testing surrobodies' capacity to inhibit the interaction between VEGF and its receptors. FIG. 2F illustrates ELISA-binding results for RO-101 or Faricimab's inhibition of the interaction between VEGF₁₆₅ and its receptors.

FIGS. 3A-3C illustrate surrobody RO-101's binding to ANG-2 and its inhibition of the interaction between ANG-2 and its receptors in comparison with Faricimab. FIG. 3A illustrates ELISA-binding results for RO-101 or Faricimab's binding to ANG-2. FIG. 3B illustrates a diagram of an assay testing surrobodies' capacity to inhibit the interaction between ANG-2 and its receptors. FIG. 3C illustrates ELISA-binding results for RO-101 or Faricimab's inhibition of the interaction between ANG-2 and its receptors.

FIGS. 4A-4B illustrate different forms of RO-101's binding to VEGF and ANG-2. FIG. 4A illustrates ELISA-binding results for binding between RO-101 SgG, scSv, or Sab that was derived from RO-101 and VEGF. FIG. 4B illustrates ELISA-binding results for binding between RO-101 SgG, scSv, or Sab that was derived from RO-101 and ANG-2.

FIGS. 5A-5D illustrate RO-101's binding to other PDGF/VEGF family members. FIG. 5A illustrates a sequence alignment among VEGF_B, VEGF_C, VEGF_D, PIGF-1, and PIGF-3. FIG. 5B illustrates ELISA-binding results for RO-101's binding to VEGF_B from different vendors in comparison with VEGF₁₆₅. FIG. 5C illustrates RO-101's binding to VEGF_C or VEGF_D in comparison with VEGF₁₆₅. FIG. 5D illustrates RO-101's binding to PIGF-1 or PIGF-3 in comparison with VEGF₁₆s.

FIGS. 6A-6D illustrate RO-101's binding to non-human orthologs for VEGF or ANG-2. FIG. 6A illustrates a sequence alignment among VEGF orthologs from human, rabbits, dogs and pigs. FIG. 6B illustrates ELISA-binding results for RO-101's binding to VEGF orthologs from human, rabbits, dogs and pigs. FIG. 6C illustrates a sequence alignment among ANG-2 orthologs from human, rabbits, dogs and pigs FIG. 6D illustrates ELISA-binding results for RO-101's binding to ANG-2 orthologs from human, rabbits, and dogs.

### DETAILED DESCRIPTION

Described herein in one aspect, is a vascular endothelial growth factor (VEGF) binding antibody or antigen binding fragment thereof comprising: a heavy chain complementarity determining region 1 (HCDR1) comprising the amino acid sequence set forth in SEQ ID NO: 3 (GYTFSIYT); a heavy chain complementarity determining region 2 (HCDR2) comprising the amino acid sequence set forth in SEQ ID NO: 4 (INPYNGNT); and a heavy chain complementarity determining region 3 (HCDR3) comprising the amino acid sequence set forth in SEQ ID NO: 5 (AKAPAVFWWTGLDY); wherein the antibody binds to VEGF.

Described herein in another aspect is an angiopoietin 2 (ANG-2) binding antibody or antigen binding fragment thereof comprising: a heavy chain complementarity determining region 1 (HCDR1) comprising the amino acid sequence set forth in SEQ ID NO: 13 (GFTFSSYG); a heavy chain complementarity determining region 2 (HCDR2) comprising the amino acid sequence set forth in SEQ ID NO: 14 (ISADSGDK); and a heavy chain complementarity determining region 3 (HCDR3) comprising the amino acid sequence set forth in SEQ ID NO: 15 (AKEFISWIYTFDYLDY), wherein the antibody binds to ANG-2.

Described herein in another aspect is a bispecific antibody comprising: a vascular endothelial growth factor (VEGF) binding moiety comprising: a heavy chain complementarity determining region 1 (HCDR1) comprising the amino acid sequence set forth in SEQ ID NO: 3 (GYTFSIYT); a heavy chain complementarity determining region 2 (HCDR2) comprising the amino acid sequence set forth in SEQ ID NO: 4 (INPYNGNT); and a heavy chain complementarity determining region 3 (HCDR3) comprising the amino acid sequence set forth in SEQ ID NO: 5 (AKAPAVFWWTGLDY); and an angiopoietin 2 (ANG-2) binding moiety comprising: a heavy chain complementarity determining region 1 (HCDR1) comprising the amino acid sequence set forth in SEQ ID NO: 13 (GFTFSSYG); a heavy chain complementarity determining region 2 (HCDR2) comprising the amino acid sequence set forth in SEQ ID NO: 14 (ISADSGDK); and a heavy chain complementarity determining region 3 (HCDR3) comprising the amino acid sequence set forth in SEQ ID NO: 15 (AKEFISWIYTFDYLDY); wherein the bispecific antibody binds to VEGF and ANG-2.

Described herein in another aspect is a bispecific antibody comprising: (a) a first heavy chain variable region comprising an amino acid sequence comprising at least 85%, 90%, 95%, 96%, 97%, 98%, 99%, 100% identity to the amino acid sequence set forth in SEQ ID NO: 1; (b) a second heavy chain variable region comprising an amino acid sequence comprising at least 85%, 90%, 95%, 96%, 97%, 98%, 99%, 100% identity to the amino acid sequence set forth in SEQ ID NO: 11; and (c) a common light chain variable region, wherein the common light chain variable region comprises a light chain variable region comprising an amino acid sequence comprising at least 85%, 90%, 95%, 96%, 97%, 98%, 99%, 100% identity to the amino acid sequence set forth in SEQ ID NO: 21, wherein the bispecific antibody binds to VEGF and ANG-2.

Described herein in another aspect is a bispecific antibody comprising: (a) a first heavy chain comprising an amino acid sequence comprising at least 85%, 90%, 95%, 96%, 97%, 98%, 99%, 100% identity to the amino acid sequence set forth in SEQ ID NO: 2; (b) a second heavy chain comprising an amino acid sequence comprising at least 85%, 90%, 95%, 96%, 97%, 98%, 99%, 100% identity to the amino acid sequence set forth in SEQ ID NO: 12; and (c) a common light chain, wherein the common light chain comprises a light chain comprising an amino acid sequence comprising at least 85%, 90%, 95%, 96%, 97%, 98%, 99%, 100% identity to the amino acid sequence set forth in SEQ ID NO: 22, wherein the bispecific antibody binds to VEGF and ANG-2.

Described herein in another aspect is a bispecific antibody comprising: (a) a first heavy chain comprising an amino acid sequence as set forth in SEQ ID NO: 2; (b) a second heavy chain comprising an amino acid sequence as set forth in SEQ ID NO: 12; and (c) a common light chain, wherein the common light chain comprises a light chain comprising an as set forth in SEQ ID NO: 22 wherein the bispecific antibody binds to VEGF and ANG-2.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein “vascular endothelial growth factor” or “VEGF” refers to the polypeptide encoded by the VEGFA gene, which amino acid sequence is available at www.uniprot.org/uniprot/P15692. Such sequence includes post-translational modifications, naturally occurring amino acid variants, and naturally occurring splice variants that do not affect the binding of the antibodies and bispecific antibodies described herein.

As used herein “Angiopoietin 2” or “ANG-2” refers to the polypeptide encoded by the ANGPT2 gene, which amino acid sequence is available at www.uniprot.org/uniprot/015123. Such sequence includes post-translational modifications, naturally occurring amino acid variants, and naturally occurring splice variants that do not affect the binding of the antibodies and bispecific antibodies described herein.

As used herein a “surrogate light chain” refers to a light chain formed by fusion of a VpreB polypeptide and λ5 polypeptide, and can promiscuously associate with two or more structurally distinct heavy chains. Such surrogate light chains are described in U.S. Pat. Nos. 8,114,967; 10,214,580; and 8,969,082, each of which are incorporated herein in their entirety.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

Among the provided antibodies are monoclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region.

The term “antibody” herein is used in the broadest sense and includes monoclonal antibodies, bispecific antibodies, and includes intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding may be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

As described herein an “epitope” refers to the binding determinant of an antibody or fragment described herein minimally necessary for specific binding of the antibody or fragment thereof to a target antigen. When the target antigen is a polypeptide the epitope will be a continuous or discontinuous epitope. A continuous epitope is formed by one region of the target antigen, while a discontinuous epitope may be formed from two or more separate regions. A discontinuous epitope, for example, may form when a target antigen adopts a tertiary structure that brings two amino acid sequences together and forms a three-dimensional structure bound by the antibody. When the target antigen is a polypeptide the epitope will generally be a plurality of amino acids linked into a polypeptide chain. A continuous epitope may comprise 5, 6, 7, 8, 9, 10, 11, 12, 13, 14, 15, 16, 17, 18, 19, or 20 contiguous amino acids. While an epitope may comprise a contiguous polymer of amino acids, not every amino acid of the polymer may be contacted by an amino acid residue of the antibody. Such non-contacted amino acids will still comprise part of the epitope as they may be important for the structure and linkage of the contacted amino acids. The skilled artisan may determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antibody target. In some embodiments, an antibody provided herein has a dissociation constant (K_{D}) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the antibody target. The antibody target can be an anti-VEGF or anti-ANG-2 antibody. K_{D} can be measured by any suitable assay. In certain embodiments, KD can be measured using surface plasmon resonance assays (e.g., using a BIACORE®-2000, a BIACORE®-3000 or Octet).

In certain embodiments, the antibodies disclosed herein bind to VEGF_A. VEGF-A may have several different splice sites thus may have various isoforms depending on splice sites. Accordingly, in certain embodiments, the antibodies disclosed herein bind to VEGF₁₆₅. In certain embodiments, the antibodies disclosed herein bind to VEGF₁₂₁. In certain embodiments, the antibodies' binding affinity to VEGF₁₂₁ is predicative of the antibodies' efficacy in treating a disease of an eye. In certain embodiments, the antibodies disclosed herein bind to a peptide comprising a platelet-derived growth factor (PDGF) domain of VEGF_A. In some embodiments, the PDGF domain of VEGF_A comprises a fragment of VEGF_A ranging from about 40^{th}, 41^{st}, 42^{nd}, 43^{rd}, 44^{th}, 45^{th}, 46^{th}, 46^{th}, 47^{th}, 48^{th}, 49^{th} or 50^{th} to about 130^{th}, 131^{st}, 132^{nd}, 133^{rd}, 134^{th}, 135^{th}, 136^{th}, 137^{th}, 138^{th}, 139^{th}, or 140^{th} amino acids. In certain embodiments, the antibodies disclosed herein bind to a peptide comprising a VEGF_C heparin domain of VEGF_A. In some embodiments, the VEGF_C heparin domain of VEGF_A comprises a fragment of VEGF_A ranging from about 140^{th}, 141^{st}, 142^{nd}, 143^{rd}, 144^{th}, 145^{th}, 146^{th}, 147^{th}, 148^{th}, 149^{th}, or 150^{th} to about 190^{th}, 191^{st}, 192^{nd}, 193^{rd}, 194^{th}, 195^{th}, 196^{th}, 197^{th}, 198^{th}, 199^{th}, 120^{th} amino acids. In certain embodiments, the antibodies disclosed herein bind to VEGF_A from a species of human or a non-human (e.g., rabbits, canines (dogs), or porcine (pigs)).

In certain embodiments, the antibodies are anti-Human VEGF antibodies which bind to human VEGF at a K_{D} or EC₅₀ of 100, 75, 60, 50, 40, 30, or 25 picomolar or less. In certain embodiments, the antibodies are anti-Human VEGF_A antibodies which bind to human VEGF at a K_{D} or EC₅₀ of 100, 75, 60, 50, 40, 30, or 25 picomolar or less. In certain embodiments, the antibodies are anti-Human VEGF_A antibodies which bind to human VEGF at a K_{D} or EC₅₀ of 1, 2, 3, 4, 5, 6, 7, 8, 9, or 10 picomolar or more. In certain embodiments, the antibodies disclosed herein bind to VEGF_A or a fragment thereof with a similar EC₅₀ as the one between Faricimab and VEGF_A or a fragment thereof. In certain embodiments, the antibodies disclosed herein bind to VEGF_A or a fragment thereof with an EC₅₀ 10, 20, 30, 40, 50, 60, 70, 80, 90, 100, 200, 300, 400, 500 pM less than the one between Faricimab and VEGF_A or a fragment thereof.

In certain embodiments, the antibodies are anti-Human VEGF_A antibodies which inhibits an interaction of VEGF-A or a fragment thereof and a VEGF receptor. In certain embodiments, the antibodies are anti-Human VEGF_A antibodies which inhibits an interaction of VEGF-A or a fragment thereof and a VEGF receptor at an IC₅₀ of 5 nM, 4 nM, 3 nM, 2 nM, 1 nM, 900 pM, 800 pM, 700 pM, 600 pM or less. In certain embodiments, the antibodies are anti-Human VEGF_A antibodies which inhibits an interaction of VEGF-A or a fragment thereof and a VEGF receptor at an IC₅₀ of 1, 2, 3, 4, 5, 6, 7, 8, 9, or 10 picomolar or more. In certain embodiments, the antibodies disclosed herein inhibit the interaction of VEGF_A or a fragment thereof and the VEGF receptor at least 1-fold, 2-fold, 3-fold, 4-fold, 5-fold, 6-fold, 7-fold, 8-fold, 9-fold, or 10-fold stronger than Faricimab inhibits the interaction. In certain embodiments, the VEGF receptor is VEGF receptor 2/kinase insert domain receptor (KDR).

In certain embodiments, the antibodies disclosed herein bind to VEGF_B significantly less than to VEGF_A. In certain embodiments, the antibodies disclosed herein bind to VEGF_C significantly less than to VEGF_A. In certain embodiments, the antibodies disclosed herein bind to VEGF_D significantly less than to VEGF_A. In certain embodiments, the antibodies disclosed herein bind to PIGF-1 significantly less than to VEGF_A. In certain embodiments, the antibodies disclosed herein bind to PIGF-3 significantly less than to VEGF_A.

In certain embodiments, the antibodies are anti-Human ANG-2 antibodies which bind to human ANG-2 at a K_{D} or EC₅₀ of 100, 75, 60, 55, 50, 40, 30, or 25 picomolar or less. In certain embodiments, the antibodies are anti-Human ANG-2 antibodies which bind to human ANG-2 at a K_{D} or EC₅₀ of 1, 2, 3, 4, 5, 6, 7, 8, 9, or 10 picomolar or more. In certain embodiments, the antibodies bind to ANG-2 at least 10-fold, 20-fold, 30-fold, 40-fold, 50-fold, 60-fold, or 70-fold stronger than Faricimab binds to ANG-2.

In certain embodiments, the antibodies are anti-Human ANG-2 antibodies which inhibits an interaction of ANG-2 and an ANG-2 receptor. In certain embodiments, the antibodies are anti-Human VEGF_A antibodies which inhibits an interaction of ANG-2 and an ANG-2 receptor at an IC₅₀ of 5 nM, 4 nM, 3 nM, 2 nM, 1 nM, 900 pM, 800 pM, 700 pM, 600 pM or less. In certain embodiments, the antibodies are anti-Human VEGF_A antibodies which inhibits an interaction of ANG-2 and an ANG-2 receptor at an IC₅₀ of 1, 2, 3, 4, 5, 6, 7, 8, 9, or 10 picomolar or more. In some embodiments, the antibodies disclosed herein inhibit the interaction of ANG-2 and the ANG-2 receptor at least 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold stronger than Faricimab inhibits the interaction. In some embodiments, the ANG-2 receptor is a tyrosine kinase with immunoglobulin-like and EGF-like domains 2 receptor (Tie-2 receptor).

In certain embodiments, the antibodies are anti-Human VEGF/ANG-2 bispecific antibodies which bind to human VEGF at a K_{D} or EC₅₀ of 100, 75, 60, 50, 40, 30, or 25 picomolar or less. In certain embodiments, the antibodies are anti-Human VEGF/ANG-2 bispecific antibodies which bind to human VEGF_A at a K_{D} or EC₅₀ of 100, 75, 60, 50, 40, 30, or 25 picomolar or less. In certain embodiments, the antibodies are anti-Human VEGF/ANG-2 bispecific antibodies which bind to human VEGF_A at a K_{D} or EC₅₀ of 1, 2, 3, 4, 5, 6, 7, 8, 9, or 10 picomolar or more.

In certain embodiments, the antibodies are anti-Human VEGF/ANG-2 bispecific antibodies which bind to human ANG-2 at a K_{D} or EC₅₀ of 100, 75, 60, 50, 40, 30, or 25 picomolar or less. In certain embodiments, the antibodies are anti-Human VEGF/ANG-2 bispecific antibodies which bind to human ANG-2 at a K_{D} or EC₅₀ of 1, 2, 3, 4, 5, 6, 7, 8, 9, or 10 picomolar or more.

In some embodiments, the antibodies disclosed herein are IgG antibodies. In some embodiments, the antibodies disclosed herein are IgE antibodies. In some embodiments, the antibodies disclosed herein are IgM antibodies. In some embodiments, the antibodies disclosed herein are IgA antibodies. In some embodiments, the antibodies disclosed herein are Fab, F(ab)₂, single-domain antibody, or single chain variable fragment (scFv).

In some embodiments, paired with one or more surrogate light chains, the antibodies disclosed herein are surrobodies. In some embodiments, the antigen binding fragment thereof is a surrobody antigen-binding fragment (Sab) or single chain variable fragment of a surrobody (scSv).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region of an immunoglobulin is important for many important antibody functions (e.g. effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the antibodies described herein comprise the variable domains of the invention combined with constant domains comprising different Fc regions, selected based on the biological activities of the antibody for the intended use. In certain instances, Human IgGs, for example, can be classified into four subclasses, IgG1, IgG2, IgG3, and IgG4, and each these of these comprises an Fc region having a unique profile for binding to one or more of Fc7 receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fc7 receptors; IgG2 binds to FcγRIIA_{H131}, and with lower affinity to FcγRIIA_{R131} FcγRIIIA_{V158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fc7 receptors. Studies have shown that FcγRI does not bind to IgG2, and FcγRIIIB does not bind to IgG2 or IgG4. Id. In general, with regard to ADCC activity, human IgG1≥IgG3>>IgG4≥IgG2.

In some embodiments, the antibodies of this disclosure are variants that possess reduced effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In some embodiments, the antibodies of this disclosure are variants that possess increased effector functions for applications in which increased immunogenicity would be beneficial. Such antibodies can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A1 18 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus. In certain embodiments, the expression vector described herein is used in a gene therapy. Accordingly, in some embodiments, the expression vector described herein is formulated for intravenous administration. In some embodiments, the expression vector described herein is formulated for intravitreal administration.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 Sep.-Oct.; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NSO murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody or bispecific antibody described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antibody or bispecific antibody described herein integrated at a genomic location; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### VEGF Antibodies

Described herein are antibodies that bind to the VEGF protein. In some embodiments, the VEGF protein is a human VEGF protein.

In certain embodiments, described herein is a vascular endothelial growth factor (VEGF) binding antibody or antigen binding fragment thereof comprising; a heavy chain complementarity determining region 1 (HCDR1) comprising the amino acid sequence set forth in SEQ ID NO: 3 (GYTFSIYT); a heavy chain complementarity determining region 2 (HCDR2) comprising the amino acid sequence set forth in SEQ ID NO: 4 (INPYNGNT); and a heavy chain complementarity determining region 3 (HCDR3) comprising the amino acid sequence set forth in SEQ ID NO: 5 (AKAPAVFWWTGLDY); wherein the antibody binds to VEGF.

In certain embodiments, described herein is a vascular endothelial growth factor (VEGF) binding antibody or antigen binding fragment thereof comprising; a heavy chain complementarity determining region 1 (HCDR1) comprising the amino acid sequence set forth in SEQ ID NO: 3 (GYTFSIYT); a heavy chain complementarity determining region 2 (HCDR2) comprising the amino acid sequence set forth in SEQ ID NO: 4 (INPYNGNT); and a heavy chain complementarity determining region 3 (HCDR3) comprising the amino acid sequence set forth in SEQ ID NO: 5 (AKAPAVFWWTGLDY); a light chain complementarity determining region 1 (LCDR1) comprising the amino acid sequence set forth in SEQ ID NO: 23 (NDHDIGVYS); a light chain complementarity determining region 2 (LCDR2) comprising the amino acid sequence set forth in SEQ ID NO: 24 (YFSQSDK); and a light chain complementarity determining region 3 (LCDR3) comprising the amino acid sequence set forth in SEQ ID NO: 25 (AMGARSSVTH); wherein the antibody binds to VEGF.

In certain embodiments, the heavy chain variable region of the VEGF antibody comprises an amino acid sequence comprising at least 85% identity to the amino acid sequence set forth in SEQ ID NO: 1. In certain embodiments, the heavy chain variable region of the VEGF antibody comprises an amino acid sequence comprising at least 90% identity to the amino acid sequence set forth in SEQ ID NO: 1. In certain embodiments, the heavy chain variable region of the VEGF antibody comprises an amino acid sequence comprising at least 95% identity to the amino acid sequence set forth in SEQ ID NO: 1. In certain embodiments, the heavy chain variable region of the VEGF antibody comprises an amino acid sequence comprising at least 96% identity to the amino acid sequence set forth in SEQ ID NO: 1. In certain embodiments, the heavy chain variable region of the VEGF antibody comprises an amino acid sequence comprising at least 97% identity to the amino acid sequence set forth in SEQ ID NO: 1. In certain embodiments, the heavy chain variable region of the VEGF antibody comprises an amino acid sequence comprising at least 98% identity to the amino acid sequence set forth in SEQ ID NO: 1. In certain embodiments, the heavy chain variable region of the VEGF antibody comprises an amino acid sequence comprising at least 99% identity to the amino acid sequence set forth in SEQ ID NO: 1. In certain embodiments, the heavy chain variable region of the VEGF antibody comprises an amino acid sequence comprising the amino acid sequence set forth in SEQ ID NO: 1.

In certain embodiments, the heavy chain of the VEGF antibody comprises an amino acid sequence comprising at least 85% identity to the amino acid sequence set forth in SEQ ID NO: 2. In certain embodiments, the heavy chain of the VEGF antibody comprises an amino acid sequence comprising at least 90% identity to the amino acid sequence set forth in SEQ ID NO: 2. In certain embodiments, the heavy chain of the VEGF antibody comprises an amino acid sequence comprising at least 95% identity to the amino acid sequence set forth in SEQ ID NO: 2. In certain embodiments, the heavy chain of the VEGF antibody comprises an amino acid sequence comprising at least 96% identity to the amino acid sequence set forth in SEQ ID NO: 2. In certain embodiments, the heavy chain of the VEGF antibody comprises an amino acid sequence comprising at least 97% identity to the amino acid sequence set forth in SEQ ID NO: 2. In certain embodiments, the heavy chain of the VEGF antibody comprises an amino acid sequence comprising at least 98% identity to the amino acid sequence set forth in SEQ ID NO: 2. In certain embodiments, the heavy chain of the VEGF antibody comprises an amino acid sequence comprising at least 99% identity to the amino acid sequence set forth in SEQ ID NO: 2. In certain embodiments, the heavy chain of the VEGF antibody comprises an amino acid sequence comprising the amino acid sequence set forth in SEQ ID NO: 2.

In certain embodiments, the light chain variable region of the VEGF antibody comprises an amino acid sequence comprising at least 85% identity to the amino acid sequence set forth in SEQ ID NO: 21. In certain embodiments, the light chain variable region of the VEGF antibody comprises an amino acid sequence comprising at least 90% identity to the amino acid sequence set forth in SEQ ID NO: 21. In certain embodiments, the light chain variable region of the VEGF antibody comprises an amino acid sequence comprising at least 95% identity to the amino acid sequence set forth in SEQ ID NO: 21. In certain embodiments, the light chain variable region of the VEGF antibody comprises an amino acid sequence comprising at least 96% identity to the amino acid sequence set forth in SEQ ID NO: 21. In certain embodiments, the light chain variable region of the VEGF antibody comprises an amino acid sequence comprising at least 97% identity to the amino acid sequence set forth in SEQ ID NO: 21. In certain embodiments, the light chain variable region of the VEGF antibody comprises an amino acid sequence comprising at least 98% identity to the amino acid sequence set forth in SEQ ID NO: 21. In certain embodiments, the light chain variable region of the VEGF antibody comprises an amino acid sequence comprising at least 99% identity to the amino acid sequence set forth in SEQ ID NO: 21. In certain embodiments, the light chain variable region of the VEGF antibody comprises an amino acid sequence set forth in SEQ ID NO: 21.

In certain embodiments, the light chain of the VEGF antibody comprises an amino acid sequence comprising at least 85% identity to the amino acid sequence set forth in SEQ ID NO: 22. In certain embodiments, the light chain of the VEGF antibody comprises an amino acid sequence comprising at least 90% identity to the amino acid sequence set forth in SEQ ID NO: 22. In certain embodiments, the light chain of the VEGF antibody comprises an amino acid sequence comprising at least 95% identity to the amino acid sequence set forth in SEQ ID NO: 22. In certain embodiments, the light chain of the VEGF antibody comprises an amino acid sequence comprising at least 96% identity to the amino acid sequence set forth in SEQ ID NO: 22. In certain embodiments, the light chain of the VEGF antibody comprises an amino acid sequence comprising at least 97% identity to the amino acid sequence set forth in SEQ ID NO: 22. In certain embodiments, the light chain of the VEGF antibody comprises an amino acid sequence comprising at least 98% identity to the amino acid sequence set forth in SEQ ID NO: 22. In certain embodiments, the light chain of the VEGF antibody comprises an amino acid sequence comprising at least 99% identity to the amino acid sequence set forth in SEQ ID NO: 22. In certain embodiments, the light chain of the VEGF antibody comprises an amino acid sequence set forth in SEQ ID NO: 22.

In certain embodiments, the VEGF antibody binds VEGF with an EC50 of about 75 picomolar or less. In certain embodiments, the VEGF antibody binds VEGF with an EC50 of about 60 picomolar or less. In certain embodiments, the VEGF antibody binds VEGF with an EC50 of about 50 picomolar or less. In certain embodiments, the VEGF antibody binds VEGF with an EC50 of about 40 picomolar or less.

### ANG-2 Antibodies

Described herein are antibodies that bind to the ANG-2 protein. In some embodiments, the ANG-2 protein is a human ANG-2 protein.

In certain embodiments, described herein is an angiopoietin 2 (ANG-2) binding antibody or antigen binding fragment thereof comprising: a heavy chain complementarity determining region 1 (HCDR1) comprising the amino acid sequence set forth in SEQ ID NO: 13 (GFTFSSYG); a heavy chain complementarity determining region 2 (HCDR2) comprising the amino acid sequence set forth in SEQ ID NO: 14 (ISADSGDK); and a heavy chain complementarity determining region 3 (HCDR3) comprising the amino acid sequence set forth in SEQ ID NO: 15 (AKEFISWIYTFDYLDY); wherein the antibody binds to ANG-2.

In certain embodiments, described herein is an angiopoietin 2 (ANG-2) binding antibody or antigen binding fragment thereof comprising: a heavy chain complementarity determining region 1 (HCDR1) comprising the amino acid sequence set forth in SEQ ID NO: 13 (GFTFSSYG); a heavy chain complementarity determining region 2 (HCDR2) comprising the amino acid sequence set forth in SEQ ID NO: 14 (ISADSGDK); and a heavy chain complementarity determining region 3 (HCDR3) comprising the amino acid sequence set forth in SEQ ID NO: 15 (AKEFISWIYTFDYLDY); a light chain complementarity determining region 1 (LCDR1) comprising the amino acid sequence set forth in SEQ ID NO: 23 (NDHDIGVYS); a light chain complementarity determining region 2 (LCDR2) comprising the amino acid sequence set forth in SEQ ID NO: 24 (YFSQSDK); and a light chain complementarity determining region 3 (LCDR3) comprising the amino acid sequence set forth in SEQ ID NO: 25 (AMGARSSVTH); wherein the antibody binds to ANG-2.

In certain embodiments, the heavy chain variable region of the ANG-2 antibody comprises an amino acid sequence comprising at least 85% identity to the amino acid sequence set forth in SEQ ID NO: 11. In certain embodiments, the heavy chain variable region of the ANG-2 antibody comprises an amino acid sequence comprising at least 90% identity to the amino acid sequence set forth in SEQ ID NO: 11. In certain embodiments, the heavy chain variable region of the ANG-2 antibody comprises an amino acid sequence comprising at least 95% identity to the amino acid sequence set forth in SEQ ID NO: 11. In certain embodiments, the heavy chain variable region of the ANG-2 antibody comprises an amino acid sequence comprising at least 96% identity to the amino acid sequence set forth in SEQ ID NO: 11. In certain embodiments, the heavy chain variable region of the ANG-2 antibody comprises an amino acid sequence comprising at least 97% identity to the amino acid sequence set forth in SEQ ID NO: 11. In certain embodiments, the heavy chain variable region of the ANG-2 antibody comprises an amino acid sequence comprising at least 98% identity to the amino acid sequence set forth in SEQ ID NO: 11. In certain embodiments, the heavy chain variable region of the ANG-2 antibody comprises an amino acid sequence comprising at least 99% identity to the amino acid sequence set forth in SEQ ID NO: 11. In certain embodiments, the heavy chain variable region of the ANG-2 antibody comprises an amino acid sequence comprising the amino acid sequence set forth in SEQ ID NO: 11.

In certain embodiments, the heavy chain of the ANG-2 antibody comprises an amino acid sequence comprising at least 85% identity to the amino acid sequence set forth in SEQ ID NO: 12. In certain embodiments, the heavy chain of the ANG-2 antibody comprises an amino acid sequence comprising at least 90% identity to the amino acid sequence set forth in SEQ ID NO: 12. In certain embodiments, the heavy chain of the ANG-2 antibody comprises an amino acid sequence comprising at least 95% identity to the amino acid sequence set forth in SEQ ID NO: 12. In certain embodiments, the heavy chain of the ANG-2 antibody comprises an amino acid sequence comprising at least 96% identity to the amino acid sequence set forth in SEQ ID NO: 12. In certain embodiments, the heavy chain of the ANG-2 antibody comprises an amino acid sequence comprising at least 97% identity to the amino acid sequence set forth in SEQ ID NO: 12. In certain embodiments, the heavy chain of the ANG-2 antibody comprises an amino acid sequence comprising at least 98% identity to the amino acid sequence set forth in SEQ ID NO: 12. In certain embodiments, the heavy chain of the ANG-2 antibody comprises an amino acid sequence comprising at least 99% identity to the amino acid sequence set forth in SEQ ID NO: 12. In certain embodiments, the heavy chain of the ANG-2 antibody comprises an amino acid sequence comprising the amino acid sequence set forth in SEQ ID NO: 12.

In certain embodiments, the light chain variable region of the ANG-2 antibody comprises an amino acid sequence comprising at least 85% identity to the amino acid sequence set forth in SEQ ID NO: 21. In certain embodiments, the light chain variable region of the ANG-2 antibody comprises an amino acid sequence comprising at least 90% identity to the amino acid sequence set forth in SEQ ID NO: 21. In certain embodiments, the light chain variable region of the ANG-2 antibody comprises an amino acid sequence comprising at least 95% identity to the amino acid sequence set forth in SEQ ID NO: 21. In certain embodiments, the light chain variable region of the ANG-2 antibody comprises an amino acid sequence comprising at least 96% identity to the amino acid sequence set forth in SEQ ID NO: 21. In certain embodiments, the light chain variable region of the ANG-2 antibody comprises an amino acid sequence comprising at least 97% identity to the amino acid sequence set forth in SEQ ID NO: 21. In certain embodiments, the light chain variable region of the ANG-2 antibody comprises an amino acid sequence comprising at least 98% identity to the amino acid sequence set forth in SEQ ID NO: 21. In certain embodiments, the light chain variable region of the ANG-2 antibody comprises an amino acid sequence comprising at least 99% identity to the amino acid sequence set forth in SEQ ID NO: 21. In certain embodiments, the light chain variable region of the ANG-2 antibody comprises an amino acid sequence set forth in SEQ ID NO: 21.

In certain embodiments, the light chain of the ANG-2 antibody comprises an amino acid sequence comprising at least 85% identity to the amino acid sequence set forth in SEQ ID NO: 22. In certain embodiments, the light chain of the ANG-2 antibody comprises an amino acid sequence comprising at least 90% identity to the amino acid sequence set forth in SEQ ID NO: 22. In certain embodiments, the light chain of the ANG-2 antibody comprises an amino acid sequence comprising at least 95% identity to the amino acid sequence set forth in SEQ ID NO: 22. In certain embodiments, the light chain of the ANG-2 antibody comprises an amino acid sequence comprising at least 96% identity to the amino acid sequence set forth in SEQ ID NO: 22. In certain embodiments, the light chain of the ANG-2 antibody comprises an amino acid sequence comprising at least 97% identity to the amino acid sequence set forth in SEQ ID NO: 22. In certain embodiments, the light chain of the ANG-2 antibody comprises an amino acid sequence comprising at least 98% identity to the amino acid sequence set forth in SEQ ID NO: 22. In certain embodiments, the light chain of the ANG-2 antibody comprises an amino acid sequence comprising at least 99% identity to the amino acid sequence set forth in SEQ ID NO: 22. In certain embodiments, the light chain of the ANG-2 antibody comprises an amino acid sequence set forth in SEQ ID NO: 22.

### VEGF/ANG-2 Bispecific Antibodies

Described herein are bispecific antibodies that bind to the VEGF and the ANG-2 protein. In some embodiments, the VEGF and/or the ANG-2 protein is a human VEGF or ANG-2 protein. The bispecific antibodies of the current disclosure are of a common light chain format, wherein the heavy chain for the VEGF binding moiety is distinct from the heavy chain of the ANG-2 binding moiety and a common light chain that can associate with either heavy chain is used. Such formats prevent unwanted heavy and light chain parings (as there is only one type of light chain) making antibody formation more efficient during production. The resulting antibody comprises two heavy chains and two light chains arranged as a classical antibody (except that the two heavy chains form a heterodimer). To prevent unwanted heavy chain homodimerization (e.g., two anti-ANG-2 heavy chains or two anti-VEGF heavy chains) the heavy chains can further comprise mutations or substitution to the heavy chain constant regions to promote proper heterodimer formation.

The common light chain bispecific structure can comprise a first and a second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance, “knobs” into “holes”) or biochemically (e.g. electrostatic interactions) prevent coupling of the two first heavy chain molecules or two second heavy chain molecules. Exemplary knob into hole mutations can comprise T366W (EU numbering) in one heavy chain and T366S/L368A/Y407V (EU numbering) in a second heavy chain. Exemplary mutations that facilitate coupling of a first and a second heavy chain molecule are disclosed, for example in WO2009089004, U.S. Pat. No. 8,642,745, US PG-PUB: US20140322756 and “The making of bispecific antibodies” MAbs. 2017 February-March; 9(2): 182-212.

The anti-VEGF/ANG-2 bispecific antibodies may comprise one or more substitutions to reduce effector function. Such substitution result in a variant Fc molecule. In some embodiments, the variant Fc region comprises an IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(uu), per EU numbering.

In certain embodiments, described herein is a bispecific antibody comprising: a vascular endothelial growth factor (VEGF) binding moiety comprising: (a) a heavy chain complementarity determining region 1 (HCDR1) comprising the amino acid sequence set forth in SEQ ID NO: 3 (GYTFSIYT); (b) a heavy chain complementarity determining region 2 (HCDR2) comprising the amino acid sequence set forth in SEQ ID NO: 4 (INPYNGNT); and (c) a heavy chain complementarity determining region 3 (HCDR3) comprising the amino acid sequence set forth in SEQ ID NO: 5 (AKAPAVFWWTGLDY); and an angiopoietin 2 (ANG-2) binding moiety comprising: (a) a heavy chain complementarity determining region 1 (HCDR1) comprising the amino acid sequence set forth in SEQ ID NO: 13 (GFTFSSYG); (b) a heavy chain complementarity determining region 2 (HCDR2) comprising the amino acid sequence set forth in SEQ ID NO: 14 (ISADSGDK); and (c) a heavy chain complementarity determining region 3 (HCDR3) comprising the amino acid sequence set forth in SEQ ID NO: 15 (AKEFISWIYTFDYLDY); wherein the bispecific antibody binds to VEGF and ANG-2.

In certain embodiments, described herein is a bispecific antibody comprising: a vascular endothelial growth factor (VEGF) binding moiety comprising: (a) a heavy chain complementarity determining region 1 (HCDR1) comprising the amino acid sequence set forth in SEQ ID NO: 3 (GYTFSIYT); (b) a heavy chain complementarity determining region 2 (HCDR2) comprising the amino acid sequence set forth in SEQ ID NO: 4 (INPYNGNT); and (c) a heavy chain complementarity determining region 3 (HCDR3) comprising the amino acid sequence set forth in SEQ ID NO: 5 (AKAPAVFWWTGLDY); and an angiopoietin 2 (ANG-2) binding moiety comprising: (a) a heavy chain complementarity determining region 1 (HCDR1) comprising the amino acid sequence set forth in SEQ ID NO: 13 (GFTFSSYG); (b) a heavy chain complementarity determining region 2 (HCDR2) comprising the amino acid sequence set forth in SEQ ID NO: 14 (ISADSGDK); and (c) a heavy chain complementarity determining region 3 (HCDR3) comprising the amino acid sequence set forth in SEQ ID NO: 15 (AKEFISWIYTFDYLDY); a light chain complementarity determining region 1 (LCDR1) comprising: (a) the amino acid sequence set forth in SEQ ID NO: 23 (NDHDIGVYS); (b) a light chain complementarity determining region 2 (LCDR2) comprising the amino acid sequence set forth in SEQ ID NO: 24 (YFSQSDK); and (c) a light chain complementarity determining region 3 (LCDR3) comprising the amino acid sequence set forth in SEQ ID NO: 25 (AMGARSSVTH); wherein the bispecific antibody binds to VEGF and ANG-2.

In certain embodiments, described herein is a bispecific antibody comprising: a vascular endothelial growth factor (VEGF) binding moiety comprising: (a) a heavy chain complementarity determining region 1 (HCDR1) comprising the amino acid sequence set forth in SEQ ID NO: 3 (GYTFSIYT); (b) a heavy chain complementarity determining region 2 (HCDR2) comprising the amino acid sequence set forth in SEQ ID NO: 4 (INPYNGNT); and (c) a heavy chain complementarity determining region 3 (HCDR3) comprising the amino acid sequence set forth in SEQ ID NO: 5 (AKAPAVFWWTGLDY); and an angiopoietin 2 (ANG-2) binding moiety comprising: (a) a heavy chain complementarity determining region 1 (HCDR1) comprising the amino acid sequence set forth in SEQ ID NO: 13 (GFTFSSYG); (b) a heavy chain complementarity determining region 2 (HCDR2) comprising the amino acid sequence set forth in SEQ ID NO: 14 (ISADSGDK); and (c) a heavy chain complementarity determining region 3 (HCDR3) comprising the amino acid sequence set forth in SEQ ID NO: 15 (AKEFISWIYTFDYLDY); a common light chain complementarity determining region 1 (LCDR1) comprising: (a) the amino acid sequence set forth in SEQ ID NO: 23 (NDHDIGVYS); (b) a light chain complementarity determining region 2 (LCDR2) comprising the amino acid sequence set forth in SEQ ID NO: 24 (YFSQSDK); and (c) a light chain complementarity determining region 3 (LCDR3) comprising the amino acid sequence set forth in SEQ ID NO: 25 (AMGARSSVTH); wherein the bispecific antibody binds to VEGF and ANG-2.

Also described herein in, certain embodiments, is a bispecific antibody comprising: (a) a first heavy chain variable region comprising an amino acid sequence comprising at least 85%, 90%, 95%, 96%, 97%, 98%, 99%, 100% identity to the amino acid sequence set forth in SEQ ID NO: 1; (b) a second heavy chain variable region comprising an amino acid sequence comprising at least 85%, 90%, 95%, 96%, 97%, 98%, 99%, 100% identity to the amino acid sequence set forth in SEQ ID NO: 11; and (c) a common light chain variable region, wherein the common light chain variable region comprises a light chain variable region comprising an amino acid sequence comprising at least 85%, 90%, 95%, 96%, 97%, 98%, 99%, 100% identity to the amino acid sequence set forth in SEQ ID NO: 21, wherein the bispecific antibody binds to VEGF and ANG-2.

In certain embodiments, the VEGF/ANG-2 bispecific antibody comprises: (a) a first heavy chain comprising an amino acid sequence comprising at least 85% identity to the amino acid sequence set forth in SEQ ID NO: 2; (b) a second heavy chain comprising an amino acid sequence comprising at least 85% identity to the amino acid sequence set forth in SEQ ID NO: 12; and (c) a common light chain, wherein the common light chain comprises a light chain comprising an amino acid sequence comprising at least 85% identity to the amino acid sequence set forth in SEQ ID NO: 22; wherein the bispecific antibody binds to VEGF and ANG-2.

In certain embodiments, the VEGF/ANG-2 bispecific antibody comprises: (a) a first heavy chain comprising an amino acid sequence comprising at least 90% identity to the amino acid sequence set forth in SEQ ID NO: 2; (b) a second heavy chain comprising an amino acid sequence comprising at least 90% identity to the amino acid sequence set forth in SEQ ID NO: 12; and (c) a common light chain, wherein the common light chain comprises a light chain comprising an amino acid sequence comprising at least 90% identity to the amino acid sequence set forth in SEQ ID NO: 22; wherein the bispecific antibody binds to VEGF and ANG-2.

In certain embodiments, the VEGF/ANG-2 bispecific antibody comprises: (a) a first heavy chain comprising an amino acid sequence comprising at least 95% identity to the amino acid sequence set forth in SEQ ID NO: 2; (b) a second heavy chain comprising an amino acid sequence comprising at least 95% identity to the amino acid sequence set forth in SEQ ID NO: 12; and (c) a common light chain, wherein the common light chain comprises a light chain comprising an amino acid sequence comprising at least 95% identity to the amino acid sequence set forth in SEQ ID NO: 22; wherein the bispecific antibody binds to VEGF and ANG-2.

In certain embodiments, the VEGF/ANG-2 bispecific antibody comprises: (a) a first heavy chain comprising an amino acid sequence comprising at least 97% identity to the amino acid sequence set forth in SEQ ID NO: 2; (b) a second heavy chain comprising an amino acid sequence comprising at least 97% identity to the amino acid sequence set forth in SEQ ID NO: 12; and (c) a common light chain, wherein the common light chain comprises a light chain comprising an amino acid sequence comprising at least 97% identity to the amino acid sequence set forth in SEQ ID NO: 22; wherein the bispecific antibody binds to VEGF and ANG-2.

In certain embodiments, the VEGF/ANG-2 bispecific antibody comprises: (a) a first heavy chain comprising an amino acid sequence comprising at least 98% identity to the amino acid sequence set forth in SEQ ID NO: 2; (b) a second heavy chain comprising an amino acid sequence comprising at least 98% identity to the amino acid sequence set forth in SEQ ID NO: 12; and (c) a common light chain, wherein the common light chain comprises a light chain comprising an amino acid sequence comprising at least 98% identity to the amino acid sequence set forth in SEQ ID NO: 22; wherein the bispecific antibody binds to VEGF and ANG-2.

In certain embodiments, the VEGF/ANG-2 bispecific antibody comprises: (a) a first heavy chain comprising an amino acid sequence comprising at least 99% identity to the amino acid sequence set forth in SEQ ID NO: 2; (b) a second heavy chain comprising an amino acid sequence comprising at least 99% identity to the amino acid sequence set forth in SEQ ID NO: 12; and (c) a common light chain, wherein the common light chain comprises a light chain comprising an amino acid sequence comprising at least 99% identity to the amino acid sequence set forth in SEQ ID NO: 22; wherein the bispecific antibody binds to VEGF and ANG-2.

Also described herein in, certain embodiments, is a bispecific antibody comprising: (a) a first heavy chain comprising an amino acid sequence as set forth in SEQ ID NO: 2; (b) a second heavy chain comprising an amino acid sequence as set forth in SEQ ID NO: 12; and (c) a common light chain, wherein the common light chain comprises a light chain comprising an as set forth in SEQ ID NO: 22; wherein the bispecific antibody binds to VEGF and ANG-2.

### Therapeutic Methods

In certain embodiments, disclosed herein, are antibodies useful for the treatment of a disease of the eye. In certain embodiments, the disease of the eye is associated with age or another comorbidity such as diabetes mellitus. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

In certain embodiments, the antibodies can be administered to a subject in need thereof by any route suitable for the administration of antibody-containing pharmaceutical compositions, such as, for example, topically, subcutaneously, intravenously, or intravitreally. In certain embodiments, the antibody or pharmaceutical composition comprising the antibody is administered topically. In certain embodiments, the antibody or pharmaceutical composition comprising the antibody is administered subcutaneously. In certain embodiments, the antibody or pharmaceutical composition comprising the antibody is administered intravenously. In certain embodiments, the antibody or pharmaceutical composition comprising the antibody is administered intravitreally.

In certain embodiments, the antibodies are administered on a suitable dosage schedule, for example, weekly, twice weekly, monthly, twice monthly, once every two weeks, once every three weeks, or once a month, once every two months, once every three months, once every four months, once every five months, or once every six months, etc. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 1 mg/kg and about 20 mg/kg. In certain embodiments, the therapeutically acceptable amount is between about 1 mg/kg and about 10 mg/kg. In certain embodiments, the therapeutically acceptable amount is between about 5 mg/kg and about 30 mg/kg. In certain embodiments, the therapeutically acceptable amount is between about 5 mg/kg and about 20 mg/kg. Intravitreal dosage concentrations can range from 50 mg/ml to 150 mg/ml for intravitreal use. Therapeutically effective amounts include amounts sufficient to ameliorate one or more symptoms associated with the disease or affliction to be treated.

### Pharmaceutically Acceptable Excipients, Carriers, and Diluents

In certain embodiments, the anti-VEGF/ANG-2 monospecific and bispecific antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration.

In certain embodiments, described herein is a method of preparing a treatment for a disease of the eye comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### EXAMPLES

The following illustrative examples are representative of embodiments of compositions and methods described herein and are not meant to be limiting in any way.

### Example 1-Production of VEGF/ANG-2 Bispecific Surrobodies

Nucleic acid sequences encoding sequences corresponding to SEQ ID NO: 2, SEQ ID NO: 12, and SEQ ID NO: 22 were cloned into plasmid vectors to direct eukaryotic cell expression. Three types of expression plasmids were constructed. The first plasmid contained 1 copy of each surrogate light chain (SLC), VEGF-specific Knob heavy chain, and ANG-2-specific Hole heavy chain. The second plasmid contains 2 copies of SLC and 1 copy of each heavy chain in the orientation of SLC:HC-Knob:HC:Hole:SLC. The third plasmid contains 2 copies of SLC and 1 copy of each heavy chain in the orientation of SLC:SLC-:HC-Knob:HC:Hole. Products expressed and purified (“RO-101”) from 3 stable cell lines were named as Sample, 1 Sample 2, and Sample 3 accordingly.

CHO cells were cultured in CD-CHO media (Life Technologies) supplemented with 6 mM L-glutamine (Life Technologies). Cells were incubated in a shaking incubator at 36.5° C., 5% CO2, 140 rpm.

Gene expression plasmids were prepared for transfection by linearizing followed by ethanol precipitation and resuspension in EB buffer to a final concentration of 400 μg/ml. Transfections were carried out via electroporation using the Gene Pulse XCell. For each transfection, viable cells were resuspended in pre-warmed CD-CHO media to 1.43×10⁷ cells/ml. 100 μl linearized DNA at a concentration of 400 μg/ml was aliquoted into a 0.4 cm gap electroporation cuvette and 700 μl cell suspension was added. Three cuvettes of cells and DNA were electroporated at 300 V, 900 pF and immediately transferred to 30 ml pre-warmed CD-CHO supplemented with 10 ml/L SP4 to generate a stable pool. Selected stable transfectants were expanded and surrobodies were isolated from supernatants by Protein A chromatography. A secondary polishing step was performed to remove undesired fractions, i.e. aggregates. Purified proteins were then analyzed by SE-HPLC and SDS-PAGE.

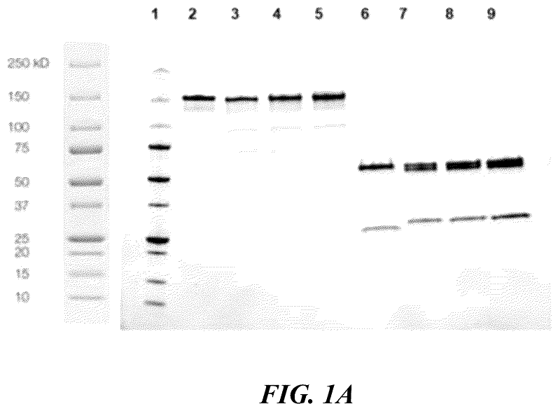

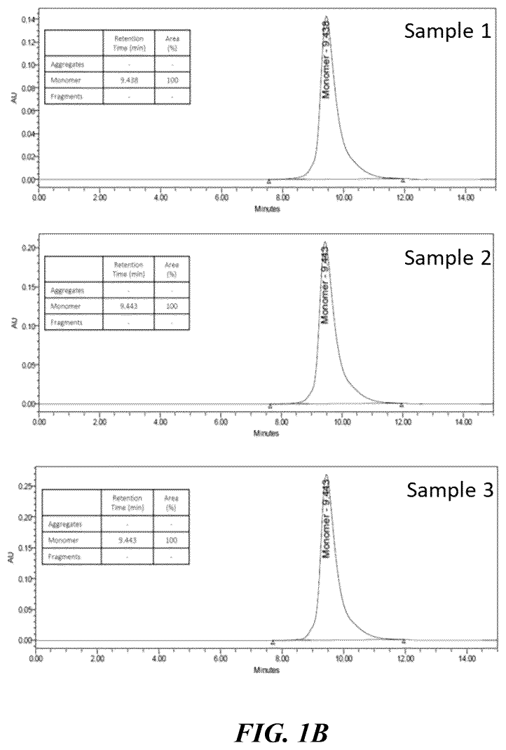

For SDS-PAGE analysis, reduced and non-reduced samples of the RO-101 were electrophoresed. FIG. 1A confirms the presence of products and good levels of purity were observed. Under non-reducing conditions, multiple protein bands between 100 and 150 kDa were seen for the products (FIG. 1A: lanes 3 to 5). Under reducing conditions, bands corresponding to the molecular weight of light chain (˜25 kDa) and heavy chains (˜50 kDa) are observed and are as expected for all three products (FIG. 1A: lanes 7 to 9). The purified RO-101 proteins were also analyzed in SE-HPLC. All three sample proteins showed 100% single peak (see FIG. 1B).

### Example 2-Binding of VEGF/ANG-2 Bispecific Surrobodies

Surrobodies RO-101 prepared according to example 1 were tested for their binding capacity to target antigen.

VEGF ELISA-Recombinant human VEGF₁₆₅ (Peprotech, Cat #100-20) was used to coat plates at 1 μg/mL. The products were diluted in blocking buffer at a starting concentration of 100 nM and then 1:3 dilutions were tested. The binding was detected by HRP-conjugated donkey anti-human IgG Fcγ antibody (Jackson ImmunoResearch Laboratories, Cat #709-035-098). Herceptin was used as a non-specific control that had no binding. Faricimab and Aflibercept were run alongside the products as reference materials. The results are shown in Table 1. The results showed high binding affinities for the products with EC50 values between 0.040 and 0.056 nM which was comparable or slightly better than binding with Faricimab and Aflibercept reference materials.

TABLE 1
EC50 for VEGF (nM)

| ELISA | | | | | | |
|---|---|---|---|---|---|---|
| Target | Herceptin | Faricimab | Aflibercept | Sample 1 | Sample 2 | Sample 3 |
| VEGF₁₆₅ | No binding | 0.04161 | 0.05135 | 0.04015 | — | — |
| | No binding | 0.05602 | 0.07402 | — | 0.05562 | — |
| | No binding | 0.05050 | 0.04025 | — | — | 0.04173 |

ANG-2 ELISA Recombinant human ANG-2 (R&D Systems, Cat #623-AN-025/CF) was used to coat plates at 1 μg/mL. The products were diluted in blocking buffer at a starting concentration of 100 nM and then 1:3 dilutions were tested. The binding was detected by HRP-conjugated donkey anti-human IgG Fcγ antibody (Jackson ImmunoResearch Laboratories, Cat #709-035-098). Herceptin was used as a non-specific control that had no binding. The results are shown in Table 2. The results showed high binding affinity for the products with EC50 values between 0.045 and 0.054 nM, while Faricimab showed significantly lower binding affinities and Aflibercept control showed no binding at all.

TABLE 2
EC50 for ANG-2 (nM)

| ELISA | | | | | | |
|---|---|---|---|---|---|---|
| Target | Herceptin | Faricimab | Aflibercept | Sample 1 | Sample 2 | Sample 3 |
| ANG-2 | No binding | 3.508 | No binding | 0.05358 | — | — |
| | No binding | 3.129 | No binding | — | 0.04984 | — |
| | No binding | 1.918 | No binding | — | — | 0.04494 |

Therefore, the tested bispecific RO-101 surrobodies showed a similar affinity to rhVEGF₁₆₅ when compared to the existing VEGF antibody or fusion protein of the extracellular domains of the human VEGFR1 and VEGFR2, and showed a much superior affinity to ANG-2 (e.g., a 42-65 fold increase in binding affinity).

### Example 3-VEGF-Binding ELISA and Inhibition of VEGF-Receptor Binding

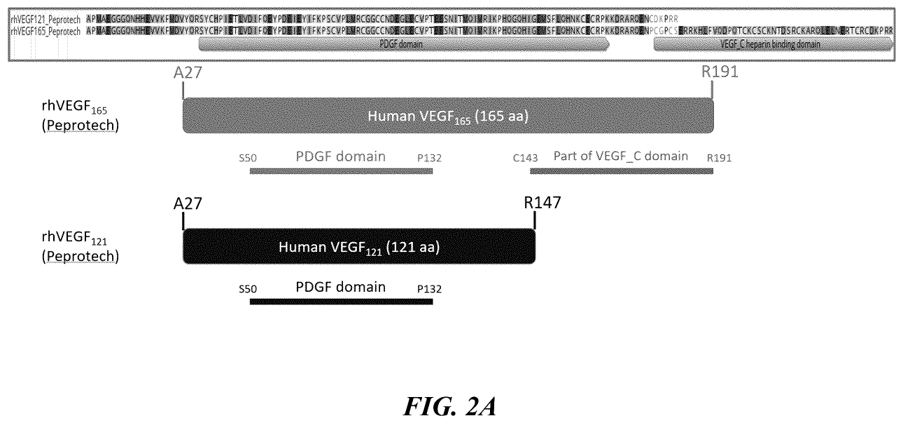

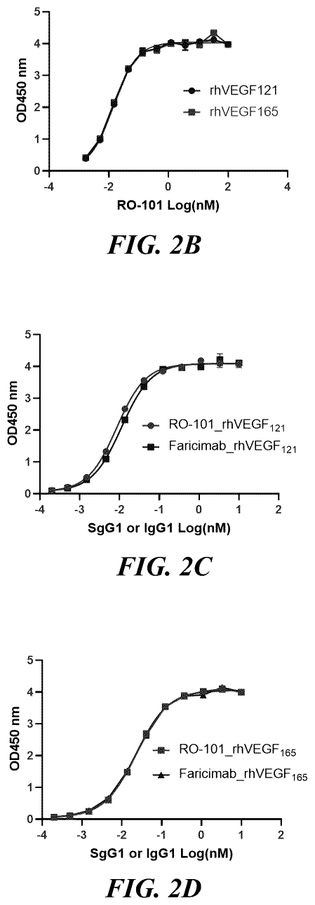

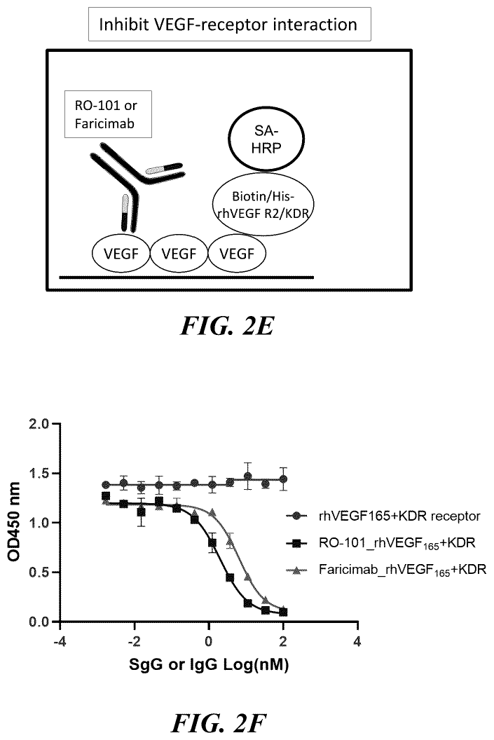

The surrobody product RO-101 was tested in terms of its binding to different isoforms of VEGF and its inhibition of VEGF-receptor interaction. VEGF₁₆₅ is the most abundant and potent isoform of VEGF-A, and VEGF₁₂₁ is the predominant isoform in eyes. Therefore, both isoforms were tested in the binding assay. Recombinant human VEGF₁₂₁ (“rh VEGF₁₂₁”) and Recombinant human VEGF₁₆₅ (“rh VEGF₁₆₅”) were obtained from Peprotech (see Table 10). The sequence alignment and analysis are shown in FIG. 2A. rhVEGF₁₂₁ contains a reserved platelet-derived growth factor (PDGF) domain without a heparin binding domain and is freely diffusible.

Accordingly, RO-101_Sample 1 binding affinities were tested against VEGF₁₂₁ and VEGF₁₆₅, respectively (see FIG. 2B). rh VEGF₁₂₁ (Peprotech, Cat #100-20A) and rh VEGF₁₆₅ (Peprotech, Cat #100-20) were used to coat Corning 9017 medium binding plates at 1 μg/mL and 100 μl per well. The products were diluted in blocking buffer at a starting concentration of 100 nM and then 1:3 dilutions were tested. Donkey anti-human IgG Fcγ-HRP (Jackson ImmunoResearch Laboratories, Cat #709-035-098) was used for detection and visualized by TMB substrate. OD450 nm readings were measured by Victor 3 (PerkinElmer 1420 Multilabel Counter).

TABLE 3
EC50 for RO-101's binding towards VEGF₁₂₁ and VEGF₁₆₅

| Target | EC50 (nM) | R² |
|---|---|---|
| rhVEGF₁₂₁ | 0.0146 | 0.9966 |
| rhVEGF₁₆₅ | 0.0135 | 0.9922 |

As shown in FIG. 2B and Table 3, RO-101 bind to rhVEGF₁₂₁ and rhVEGF₁₆₅ with a similar affinity.

Next, RO-101_Sample 1 in the format of SgG1 was compared to Faricimab (IgG1) in terms of their binding affinity towards rhVEGF₁₂₁ in an ELISA binding assay. rhVEGF₁₂₁ was used to coat Corning 9017 medium binding plates. RO-101 were diluted in blocking buffer at a starting concentration of 10 nM and then 1:3 dilutions were tested. Donkey anti-human IgG Fcγ-HRP was used for detection (Jackson ImmunoResearch Laboratories, Cat #709-035-098) and visualized by TMB substrate. OD450 nm readings were measured by Victor 3 (PerkinElmer 1420 Multilabel Counter).

TABLE 4
EC50 for RO-101 or Faricimab's binding towards VEGF₁₂₁

| SgG/IgG | EC50 nM | R² |
|---|---|---|
| RO-101_Sample 1 | 0.0086 | 0.9984 |
| Faricimab | 0.0113 | 0.9980 |

As shown in FIG. 2C and Table 4, RO-101 and Faricimab bind to rhVEGF₁₂₁ with a similar affinity in ELISA.

Next, RO-101 sample 1 was compared to Faricimab in terms of their binding affinity towards rhVEGF₁₆₅ in an ELISA binding assay. VEGF₁₆₅ was used to coat Corning 9017 medium binding plates. RO-101 were diluted in blocking buffer at a starting concentration of 10 nM and then 1:3 dilutions were tested. Donkey anti-human IgG Fcγ-HRP (Jackson ImmunoResearch Laboratories, Cat #709-035-098) was used for detection and visualized by TMB substrate. OD450 nm readings were measured by Victor 3 with PerkinElmer 1420 Multilabel Counter.

TABLE 5
EC50 for RO-101 or Faricimab's binding towards VEGF₁₆₅

| SgG/IgG | EC50 nM | R² |
|---|---|---|
| RO-101_Sample 1 | 0.0231 | 0.9995 |
| Faricimab | 0.0228 | 0.9989 |

As shown in FIG. 2D and Table 5, RO-101 and Faricimab bind to rhVEGF₁₆₅ with a similar affinity in ELISA.

Next, RO-101_Sample 1 was compared to Faricimab in terms of their inhibition of the interaction between VEGF₁₆₅ and VEGF receptor 2 (VEGF R2). VEGF R2 is also known as kinase insert domain receptor (KDR). As shown in FIG. 2E, 1:3 serial diluted RO-101 or Faricimab starting at 200 nM were mixed with 2 nM biotinylated rhVEGF R2/KDR receptor (AcroBiosystems, Cat #KDR-H82E5) at 1:1 ratio. The mixture was added to ELISA binding plates which contained rhVEGF₁₆₅ coating. Biotinylated rhVEGF R2/KDR without RO-101 or Farcimab was used as the binding controls. The receptor binding was detected by HRP-conjugated streptavidin (Jackson ImmunoResearch Laboratories, Cat #016-030-084) and visualized by TMB substrate. The results are shown in FIG. 2F and the corresponding IC50 values are shown in Table 6.

TABLE 6
IC50 for RO-101 or Faricimab's inhibition
of VEGF₁₆₅-VEGF R2/KDR receptor binding

| SgG/IgG Inhibition | IC50 nM | R² | Fold |
|---|---|---|---|
| RO-101_Sample 1 | 1.989 | 0.9863 | 3.25x |
| Faricimab | 6.471 | 0.9919 | |

As shown in FIG. 2F and Table 6, RO-101_Sample 1 was more than 3 fold stronger than Faricimab in inhibiting the interaction between rhVEGF₁₆₅ and rhVEGF R2/KDR receptor.

### Example 4-ANG-2-Binding ELISA and Inhibition of ANG-2-Receptor Binding

The Surrobody product RO-101 was tested in terms of its binding to ANG-2 and its inhibition of the interaction between ANG-2 and its receptors.

RO-101_Sample 1 or RO-101 in a concentrated solution (“RO-101 LX”) was compared to Faricimab in terms of their binding affinity towards rhANG-2 in an ELISA binding assay. rhANG-2 (AcroBioSystems, Cat #AN2-H52H4) was used to coat Corning 9017 medium binding plates. RO-101 were diluted in blocking buffer at a starting concentration of 100 nM and then 1:3 dilutions were tested. Donkey anti-human IgG Fcγ-HRP (Jackson ImmunoResearch Laboratories, Cat #709-035-098) was used for detection and visualized by TMB substrate. OD450 nm readings were measured by Victor 3 with PerkinElmer 1420 Multilabel Counter.

TABLE 7
EC50 for RO-101 or Faricimab's binding towards ANG-2

| SgG/IgG Starting Conc. | EC50 nM | R² | RO-101/Faricimab Fold |
|---|---|---|---|
| RO-101_Sample 1 | 0.0118 | 0.9940 | 53-fold |
| RO-101_LX | 0.0138 | 0.9961 | |
| Faricimab | 0.6764 | 0.9989 | |

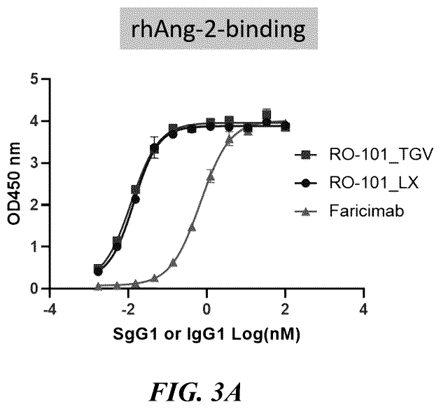

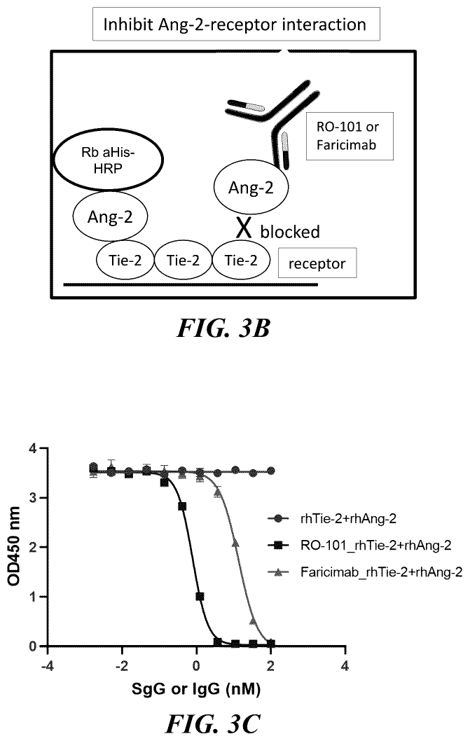

As shown in FIG. 3A and Table 7, the binding affinity of RO-101 to rhANG-2 is 53 fold stronger than Faricimab in ELISA. This was a significant improvement to Faricimab by increasing the binding to one of the two antigens.

Importantly, a similar ELISA binding experiment was carried out by an independent team and similar results were obtained. EC50 values for RO-101 were 0.0526, 0.0498, and 0.0449 nM, with an average value of 0.0491 nM. On the other hand, EC50 values for Faricimab were 3.508, 3.129, and 1.918 nM, with an average value of 2.8517 nM. Therefore, RO-101 binding of rhANG-2 was 58 fold better than Faricimab in a different experiment setting. This further confirmed that the binding affinity of RO-101 to ANG-2 is much better than Faricimab.

Next, RO-101_Sample 1 was compared to Faricimab in terms of their inhibition of the interaction between human ANG-2 and tyrosine kinase with immunoglobulin-like and EGF-like domains 2 receptor (Tie-2), an ANG-2 receptor. 1:3 serial diluted RO-101 or Faricimab starting at 200 nM were mixed with 2 nM His-tagged rhANG-2 (AcroBiosystems, Cat #AN2-H52H4) at 1:1 ratio.rhANG-2-His without RO-101 or Faricimab served as the binding control. As shown in FIG. 3B, RO-101 or Faricimab was added to ELISA binding plates which had a recombinant human Tie-2-Fc coating (AcroBiosystems, Cat #TI2-H5255). The binding of rhANG-2 to rhTie-2-Fc was detected by HRP-conjugated rabbit anti-His antibody (Bethyl Laboratories, Cat #A190-114P). The results are shown in FIG. 3C and the corresponding IC50 values are shown in Table 8.

TABLE 8
IC50 for RO-101 or Faricimab's inhibition
between ANG-2 and ANG-2 receptor binding

| SgG/IgG Inhibition | IC50 nM | R² | Fold |
|---|---|---|---|
| RO-101_Sample 1 | 0.7868 | 0.9989 | 17.2x |
| Faricimab | 13.55 | 0.9956 | |

As shown in FIG. 3C and Table 8, RO-101_Sample 1 was more than 17 fold stronger than Faricimab in inhibiting the interaction between ANG-2 and Tie-2 receptor.

Taken together, RO-101 exhibited superior binding characteristics to rhANG-2 when compared to Faricimab.

### Example 5—Binding Affinity of Different Forms of RO-101

VEGF-A specific surrobody antigen binding monomerics (Sab), a Fab equivalence, and single-chain fragments (scSv), a scFv equivalence, derived from RO-101 were expressed in HEK293 cells. Specifically, Sab, a 50 kDa form of the surrobody, comprised a VH-CH1 domain associated to VpreB and lambda 5 (surrogate light chain). scSV comprised a single chain fragment comprised of VH domain linked to VpreB.

The resulting supernatants were purified by CaptureSelect IgG-CH1 or Ni-NTA for His tagged scSv protein. Together with RO-101 SgG, the three forms were tested in terms of their binding affinity towards rhVEGF₁₆₅ and rhANG-2, respectively.

RO-101 SgG1, Sab, and scSv were tested in duplicates. They were diluted in blocking buffer at a starting concentration of 10 nM and then 1:3 dilutions were tested. rhVEGF₁₆₅-binding ELISA was detected by biotinylated anti-human VpreB1 antibody and HRP-conjugated streptavidin.

TABLE 9.1
EC50 for RO-101 SgG, Sab, and scSv's binding
towards rhVEGF₁₆₅ detected by biotinylated anti-
VpreB antibody and HRP-conjugated streptavidin

| Test Protein | EC50 (nM) |
|---|---|
| RO-101_SgG | 0.0057 |
| RP-008_VEGF-A_scSv | 0.009 |
| RP-014_VEGF-A_Sab | 0.0063 |

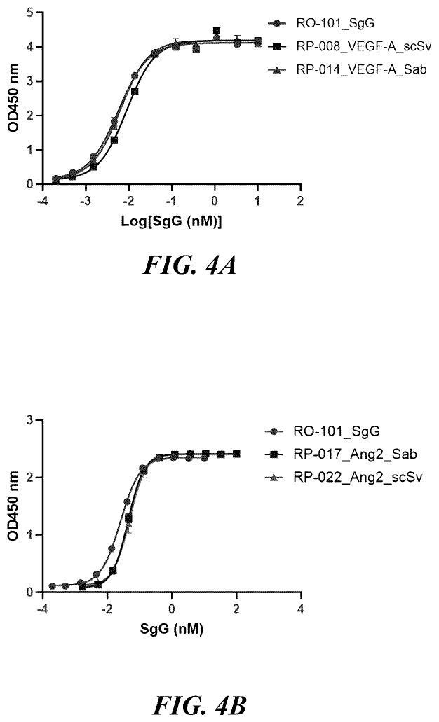

As shown in FIG. 4A and Table 9.1, monomeric anti-VEGF-A Sab and scSv bind well to rhVEG₁₆₅, to an extent that was comparable to its SgG form. Therefore, all three forms of VEGF-A-specific surrobody had similar binding affinity towards rhVEGF₁₆₅.

RO-101 SgG1, Sab, and scSv were tested in duplicates. SgG1 was diluted in blocking buffer at a starting concentration of 10 nM. Sab and scSv were diluted in blocking buffer at a starting concentration of 100 nM. They were then 1:3 serial diluted. ANG-2-binding ELISA was detected by biotinylated anti-human VpreB1 antibody and HRP-conjugated streptavidin.

TABLE 9.2
EC50 for RO-101 SgG, Sab, and scSv's binding
towards ANG-2 detected by biotinylated anti-
VpreB antibody and HRP-conjugated streptavidin

| Test Protein | EC50 (nM) |
|---|---|
| RO-101_SgG | 0.0257 |
| RP-022_ANG-2_scSv | 0.0442 |
| RP-017_ANG-2_Sab | 0.0485 |

As shown in FIG. 4B and Table 9.2, monomeric anti-ANG-2 Sab and scSv bind well to ANG-2, with picomolar affinity to rhANG-2.

### Example 6—Binding of RO-101 to PDGF/VEGF Family Members

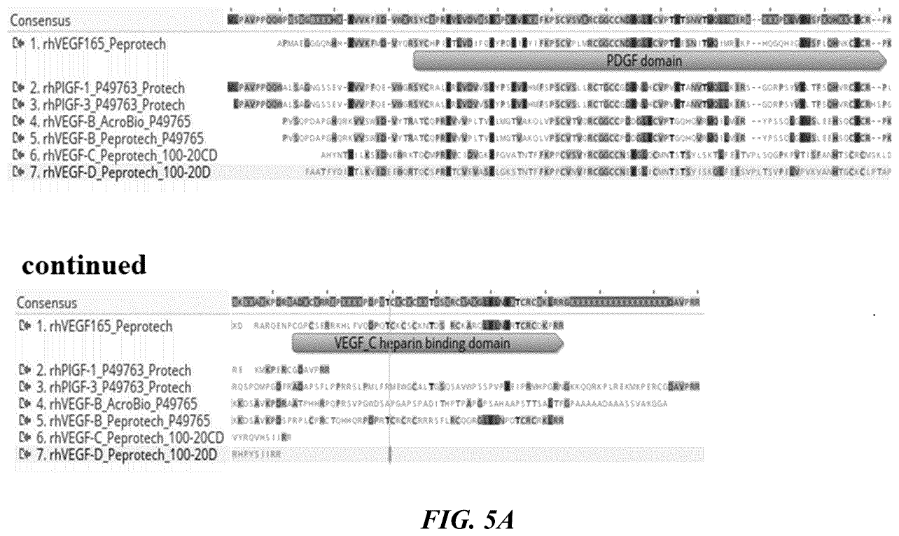

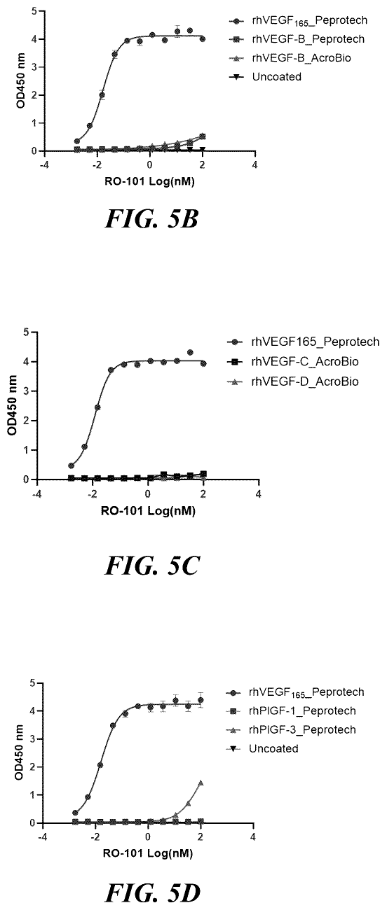

PDGF/VEGF family members were obtained from three different vendors: Peprotech, R&D Systems, and AcroBiosystems (see Table 10). Sequence alignment and analysis were conducted (see FIG. 5A). Specifically, rhVEGF-B from Peprotech and R&D had identical sequences, while the rhVEGF-B sequence from AcroBiosystems had a different C terminal. All three vendors used the same rhVEGF-C sequence, except that AcroBiosystems' rhVEGF-C sequence had additional 9 residues at the N terminus. All three vendors used the same rhVEGF-D sequence, except that Peprotech's rhVEGF-D sequence had additional 4 residues at the N terminus and 4 residues at the C terminus. The RO-101's cross-reactivity to two additional human PDGF/VEGF family members, placenta growth factor-1 (PlGF-1) and placenta growth factor-3 (PlGF-3) containing the conserved cysteine-knot domain, was also examined.

TABLE 10
summary of basic information for PDGF/VEGF
family members that were tested

| Target | Vendor_Cat. No. | Source |
|---|---|---|
| rhVEGF₁₆₅ | Peprotech_100-20 | E. coli |
| rhVEGF₁₂₁ | Peprotech_100-20A | E. coli |
| rhVEGF-B_Peprotech | Peprotech_100-20B | E. coli |
| rhVEGF-B_AcroBio | AcroBio_VE6-H5225 | His-tagged; HEK293 |
| rhVEGF_C | AcroBio_VEC-H4225 | His-tagged; HEK293 |
| rhVEGF-D | AcroBio_VED-H5228 | His-tagged; HEK293 |
| rhPlGF-1 | Peprotech_100-06 | E. coli |
| rhPlGF-3 | Peprotech_100-57 | E. coli |

First, the binding to rhVEGF-B was tested in comparison with rhVEGF₁₆₅. rhVEGF-B or rhVEGF₁₆₅ was used to coat Corning 9017 medium binding plates at 1 μg/mL and 100p per well. The products RO-101_sample 1 were diluted in blocking buffer at a starting concentration of 100 nM and then 1:3 dilutions were tested. Donkey anti-human IgG Fcγ-HRP was used for detection. OD450 nm readings were measured by Victor 3.

TABLE 11
EC50 for RO-101_Sample 1's
binding towards rhVEGF₁₆₅ or rhVEGF-B

| Target | RO-101_Sample 1 (EC50 nM) |
|---|---|
| rhVEGF₁₆₅ | 0.0165 |
| rhVEGF-B_Peprotech | >>>100 |
| rhVEGF-B_AcroBio | >>>100 |

As shown in FIG. 5B and Table 11, RO-101 had little binding to rhVEGF-B at 100 nM.

Second, RO-101_Sample 1's binding to rhVEGF-C or rhVEGF-D was tested in comparison with rhVEGF₁₆₅. rhVEGF-C and rhVEGF-D were obtained from AcroBiosystems. rhVEGF₁₆₅ was obtained from Peprotech, which served as a positive control. Different target proteins were used to coat Corning 9017 medium binding plates at 1 μg/mL and 100 μl per well. Donkey anti-human IgG Fcγ-HRP was used for detection. OD450 nm readings were measured by Victor 3.

TABLE 12
EC50 for RO-101_Sampel 1's binding
towards rhVEGF₁₆₅, rhVEGF-C or rhVEGF-D

| Target | RO-101_Sample 1 (EC50 nM) |
|---|---|
| rhVEGF₁₆₅ | 0.01223 |
| rhVEGF-C | Not bind |
| rhVEGF-D | Not bind |

As shown in FIG. 5C and Table 12, RO-101 did not bind to commercial rhVEGF-C or rhVEGF-D proteins which contain PDGF domain only.

Third, the binding to rhPIGF-1 or rhPIGF-3 was tested in comparison with rhVEGF₁₆₅. Target proteins were used to coat Corning 9017 medium binding plates at 1 μg/mL and 100 μl per well. The products RO-101_sample 1 were diluted in blocking buffer at a starting concentration of 100 nM and then 1:3 dilutions were tested. Donkey anti-human IgG Fcγ-HRP was used for detection. OD450 nm readings were measured by Victor 3.

TABLE 13
EC50 for RO-101_Sample 1's binding
towards rhVEGF₁₆₅, rhPlGF-1 or rhPlGF-3

| Target | RO-101_Sample 1 (EC50 nM) |
|---|---|
| rhVEGF₁₆₅ | 0.0162 |
| rhPlGF-1_Peprotech | Not bind |
| rhPlGF-3 Peprotech | >100 |

As shown in FIG. 5D and Table 13, RO-101 had no binding to PIGF-1 and weak binding to PIGF-3 at >100 nM.

### Example 7—Binding of RO-101 to Cross-Species VEGF-A and ANG-2

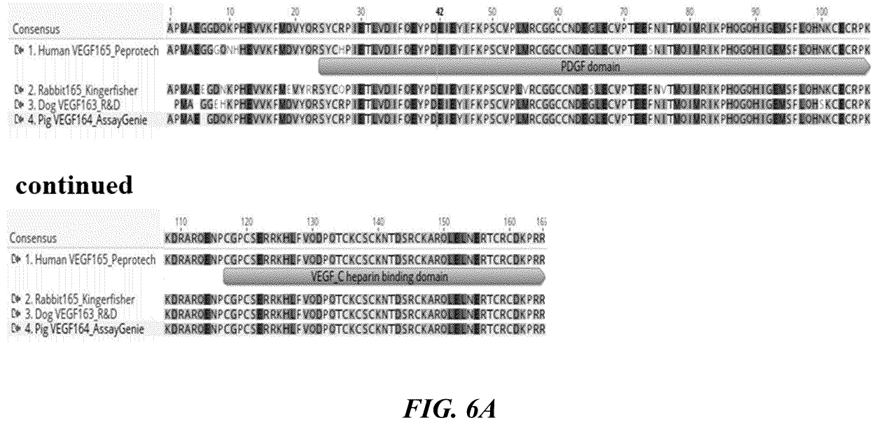

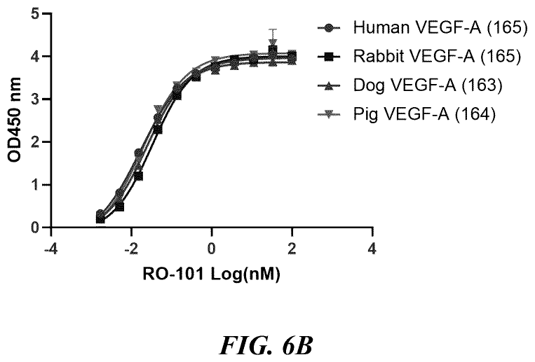

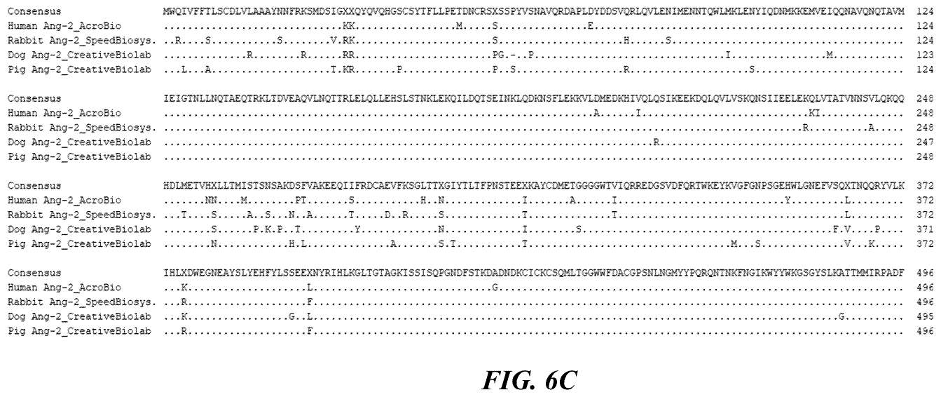

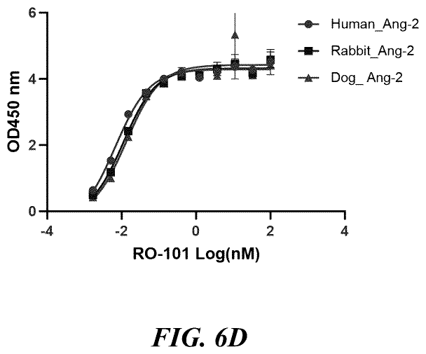

RO-101 was tested for its affinity to VEGF₁₆₅ and ANG-2 orthologs from different species (see Table 14). Sequence alignment (see FIG. 6A for VEGF and FIG. 6C for ANG-2) and homology comparison (see Table 15.1 for VEGF and Table 15.2 for ANG-2) were conducted.

TABLE 14
summary of basic information for VEGF₁₆₅ and
ANG-2 orthologs from different species

| Target | Species | Vendor_Cat No. | Source |
|---|---|---|---|
| rhVEGF₁₆₅ | Human | Peprotech_100-20 | E. coli |
| rrVEGF₁₆₅ | Rabbit | Kingfisher | Yeast |
| | | Biotech_RP1025U-025 | |
| rdVEGF₁₆₃ | Canine/Dog | R&D 1603-CV-010/CF | E. coli |
| rpVEGF₁₆₄ | Porcine/Pig | AssayGenie_KPRT0149 | Yeast |
| rhANG-2 | Human | AcroBio_AN2-H52H4 | HEK293 |
| rrANG-2 | Rabbit | Speed | HEK293 |
| | | Biosystems_YCP8015 | |
| rdANG-2 | Canine/Dog | Producing @Creative | Mammalian cell |
| | | Biolab_Vang-3062Lsx | |

TABLE 15.1
percentage of homology for
VEGF₁₆₅ orthologs from different species

| | Human | Rabbit | Dog | Pig |
|---|---|---|---|---|
| % Identity | VEGF-A | VEGF-A | VEGF-A | VEGF-A |
| Human VEGF-A | 100 | 92.72 | 95.12 | 96.36 |
| Rabbit VEGF-A | 92.72 | 100 | 93.29 | 95.15 |
| Dog VEGF-A | 95.12 | 93.29 | 100 | 96.95 |
| Pig VEGF-A | 96.36 | 95.15 | 96.95 | 100 |

TABLE 15.2
percentage of homology for ANG-2
orthologs from different species

| | Human | Rabbit | Dog | Pig |
|---|---|---|---|---|
| % Identity | ANG-2 | ANG-2 | ANG-2 | ANG-2 |
| Human ANG-2 | 100 | 92.54 | 92.34 | 92.74 |
| Rabbit ANG-2 | 92.54 | 100 | 91.734 | 93.55 |
| Dog ANG-2 | 92.34 | 91.734 | 100 | 92.54 |
| Pig ANG-2 | 92.74 | 93.55 | 92.54 | 100 |

Similar binding assays were performed as described in the previous examples. FIG. 6B and Table 16 show that RO-101 bind to rabbit, dog, and pig VEGF-A targets with a comparable affinity when compared to the binding of human VEGF-A.

TABLE 16
EC50 for RO-101's binding towards
VEGF-A from different species

| X-species VEGF-A | EC50 (nM) | R² |
|---|---|---|
| Human VEGF-A | 0.0171 | 0.9967 |
| Rabbit VEGF-A | 0.0334 | 0.9967 |
| Dog VEGF-A | 0.0237 | 0.9979 |
| Pig VEGF-A | 0.0198 | 0.9899 |

Similarly, FIG. 6D and Table 17 show that RO-101 bind to rabbit, and dog ANG-2 targets with a comparable affinity to human ANG-2.

TABLE 17
EC50 for RO-101's binding towards ANG-2 from different species

| X-species ANG-2 | EC50 (nM) | R² |
|---|---|---|
| Human ANG-2 | 0.007 | 0.9814 |
| Rabbit ANG-2 | 0.011 | 0.9868 |
| Dog ANG-2 | 0.0135 | 0.9376 |

Taken together, RO-101 exhibited similarly strong binding affinity to VEGF and ANG-2 orthologs across several species.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequences described herein

| | | |
|---|---|---|
| 1 | QVQLVQSGAEVKKPGASVKVSCKASGYTFSIYTIHWVRQAP | VEGF VH |
| | GQGLEWMGGINPYNGNTKYAQKFQGRVTMTRDTSISTAY | |
| | MELSRLRSDDTAVYYCAKAPAVFWWTGLDYWGQGTLVTV | |
| | SS | |
| 2 | QVQLVQSGAEVKKPGASVKVSCKASGYTFSIYTIHWVRQAP | VEGF HC |
| | GQGLEWMGGINPYNGNTKYAQKFQGRVTMTRDTSISTAY | |
| | MELSRLRSDDTAVYYCAKAPAVFWWTGLDYWGQGTLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSCDKTHTCPPCPAPEAAGGPSV | |
| | FLFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVYTLPPCRDELTKNQ | |
| | VSLWCLVKGFYPSDIAVEWESNGQPENNYKTTPPVLDSDGS | |
| | FFLYSKLTVDKSRWQQGNVFSCSVMHEALHNHYTQKSLSL | |
| | SPG | |
| 3 | GYTFSIYT | VEGF HCDR1 |
| 4 | INPYNGNT | VEGF HCDR2 |
| 5 | AKAPAVFWWTGLDY | VEGF HCDR3 |
| 11 | EVQLVESGGGLVKPGGSLRLSCAASGFTFSSYGMHWVRQA | Ang-2 VH |
| | PGKGLEWVAGISADSGDKNYADSVKGRFTISRDNAKNSLYL | |
| | QMNSLRAEDTAVYYCAKEFISWIYTFDYLDYWGQGTLVTV | |
| | SS | |
| 12 | EVQLVESGGGLVKPGGSLRLSCAASGFTFSSYGMHWVRQA | Ang-2 HC |
| | PGKGLEWVAGISADSGDKNYADSVKGRFTISRDNAKNSLYL | |
| | QMNSLRAEDTAVYYCAKEFISWIYTFDYLDYWGQGTLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSCDKTHTCPPCPAPEAAGGPSV | |
| | FLFPPKPKDTLMISRTPEVTCVVVDVSHEDPEVKFNWYVDG | |
| | VEVHNAKTKPREEQYNSTYRVVSVLTVLHQDWLNGKEYK | |
| | CKVSNKALPAPIEKTISKAKGQPREPQVCTLPPSRDELTKNQ | |
| | VSLSCAVKGFYPSDIAVEWESNGQPENNYKTTPPVLDSDGS | |
| | FFLVSKLTVDKSRWQQGNVFSCSVMHEALHNHYTQKSLSL | |
| | SPG | |
| 13 | GFTFSSYG | Ang-2 HCDR1 |
| 14 | ISADSGDK | Ang-2 HCDR2 |
| 15 | AKEFISWIYTFDYLDY | Ang-2 HCDR3 |
| 21 | QPVLHQPPAMSSALGTTIRLTCTLRNDHDIGVYSVYWYQQR | VL |
| | PGHPPRFLLRYFSQSDKSQGPQVPPRFSGSKDVARNRGYLSI | |
| | SELQPEDEAMYYCAMGARSSVTHVFGSGTQLTV | |
| 22 | QPVLHQPPAMSSALGTTIRLTCTLRNDHDIGVYSVYWYQQR | LC |
| | PGHPPRFLLRYFSQSDKSQGPQVPPRFSGSKDVARNRGYLSI | |
| | SELQPEDEAMYYCAMGARSSVTHVFGSGTQLTVLSQPKAT | |
| | PSVTLFPPSSEELQANKATLVCLMNDFYPGILTVTWKADGT | |
| | PITQGVEMTTPSKQSNNKYAASSYLSLTPEQWRSRRSYSCQ | |
| | VMHEGSTVEKTVAPAECS | |
| 23 | NDHDIGVYS | LCDR1 |
| 24 | YFSQSDK | LCDR2 |
| 25 | AMGARSSVTH | LCDR3 |

1.-117. (canceled)

118. A vascular endothelial growth factor A (VEGF-A) binding antibody or antigen binding fragment thereof comprising:
a. a heavy chain complementarity determining region 1 (HCDR1) comprising the amino acid sequence set forth in SEQ ID NO: 3 (GYTFSIYT);
b. a heavy chain complementarity determining region 2 (HCDR2) comprising the amino acid sequence set forth in SEQ ID NO: 4 (INPYNGNT); and
c. a heavy chain complementarity determining region 3 (HCDR3) comprising the amino acid sequence set forth in SEQ ID NO: 5 (AKAPAVFWWTGLDY);
wherein the antibody or antigen binding fragment thereof binds to VEGF-A.

119. The antibody or antigen binding fragment thereof of claim 118, comprising a heavy chain variable region comprising an amino acid sequence comprising at least 85%, 90%, 95%, 96%, 97%, 98%, 99%, or 100% identity to the amino acid sequence set forth in SEQ ID NO: 1.

120. The antibody or antigen binding fragment thereof of claim 118, comprising a heavy chain constant region comprising an amino acid sequence comprising at least 85%, 90%, 95%, 96%, 97%, 98%, 99%, or 100% identity to the amino acid sequence set forth in SEQ ID NO: 2.

121. The antibody or antigen binding fragment thereof of claim 118, further comprising a surrogate light chain, and wherein the surrogate light chain comprises:
a. a light chain complementarity determining region 1 (LCDR1) comprising the amino acid sequence set forth in SEQ ID NO: 23 (NDHDIGVYS);
b. a light chain complementarity determining region 2 (LCDR2) comprising the amino acid sequence set forth in SEQ ID NO: 24 (YFSQSDK); and
c. a light chain complementarity determining region 3 (LCDR3) comprising the amino acid sequence set forth in SEQ ID NO: 25 (AMGARSSVTH).

122. A nucleic acid or plurality of nucleic acids encoding the antibody or antigen binding fragment thereof of claim 118.

123. A pharmaceutical composition comprising the antibody or antigen binding fragment thereof of claim 118 and a pharmaceutically acceptable carrier, excipient, or diluent.

124. A method of treating a disease of an eye in an individual comprising administering to the antibody or antigen binding fragment thereof of claim 118, thereby treating the disease of the eye.

125. An angiopoietin 2 (ANG-2) binding antibody or antigen binding fragment thereof comprising:
a. a heavy chain complementarity determining region 1 (HCDR1) comprising the amino acid sequence set forth in SEQ ID NO: 13 (GFTFSSYG);
b. a heavy chain complementarity determining region 2 (HCDR2) comprising the amino acid sequence set forth in SEQ ID NO: 14 (ISADSGDK); and
c. a heavy chain complementarity determining region 3 (HCDR3) comprising the amino acid sequence set forth in SEQ ID NO: 15 (AKEFISWIYTFDYLDY),
wherein the antibody or antigen binding fragment thereof binds to ANG-2.

126. The antibody or antigen binding fragment thereof of claim 125, comprising a heavy chain variable region comprising an amino acid sequence comprising at least 85%, 90%, 95%, 96%, 97%, 98%, 99%, or 100% identity to the amino acid sequence set forth in SEQ ID NO: 11.

127. The antibody or antigen binding fragment thereof of claim 125, comprising a heavy chain constant region comprising an amino acid sequence comprising at least 85%, 90%, 95%, 96%, 97%, 98%, 99%, or 100% identity to the amino acid sequence set forth in SEQ ID NO: 12.

128. The antibody or antigen binding fragment thereof of claim 125, further comprising a surrogate light chain, and wherein the surrogate light chain comprises:
a. a light chain complementarity determining region 1 (LCDR1) comprising the amino acid sequence set forth in SEQ ID NO: 23 (NDHDIGVYS);
b. a light chain complementarity determining region 2 (LCDR2) comprising the amino acid sequence set forth in SEQ ID NO: 24 (YFSQSDK); and
c. a light chain complementarity determining region 3 (LCDR3) comprising the amino acid sequence set forth in SEQ ID NO: 25 (AMGARSSVTH).

129. A nucleic acid or plurality of nucleic acids encoding the antibody or antigen binding fragment thereof of claim 125.

130. A pharmaceutical composition comprising the antibody or antigen binding fragment thereof of claim 125 and a pharmaceutically acceptable carrier, excipient, or diluent.

131. A method of treating a disease of an eye in an individual comprising administering to the antibody or antigen binding fragment thereof of claim 125, thereby treating the disease of the eye.

132. A multispecific antibody comprising:
a vascular endothelial growth factor A (VEGF-A) binding moiety comprising:
a. a heavy chain complementarity determining region 1 (HCDR1) comprising the amino acid sequence set forth in SEQ ID NO: 3 (GYTFSIYT);
b. a heavy chain complementarity determining region 2 (HCDR2) comprising the amino acid sequence set forth in SEQ ID NO: 4 (INPYNGNT); and
c. a heavy chain complementarity determining region 3 (HCDR3) comprising the amino acid sequence set forth in SEQ ID NO: 5 (AKAPAVFWWTGLDY); and
an angiopoietin 2 (ANG-2) binding moiety, wherein the multispecific antibody binds to VEGF-A and ANG-2.

133. The multispecific antibody of claim 132, wherein the ANG-2 binding moiety comprises:
a. a heavy chain complementarity determining region 1 (HCDR1) comprising the amino acid sequence set forth in SEQ ID NO: 13 (GFTFSSYG);
b. a heavy chain complementarity determining region 2 (HCDR2) comprising the amino acid sequence set forth in SEQ ID NO: 14 (ISADSGDK); and
c. a heavy chain complementarity determining region 3 (HCDR3) comprising the amino acid sequence set forth in SEQ ID NO: 15 (AKEFISWIYTFDYLDY).

134. The multispecific antibody of claim 132, wherein the VEGF-A binding moiety comprises a heavy chain variable region comprising an amino acid sequence at least 85%, 90%, 95%, 96%, 97%, 98%, 99%, or 100% identity to the amino acid sequence set forth in SEQ ID NO: 1.

135. The multispecific antibody of claim 132, wherein the VEGF-A binding moiety comprises a heavy chain constant region comprising an amino acid sequence comprising at least 85%, 90%, 95%, 96%, 97%, 98%, 99%, or 100% identity to the amino acid sequence set forth in SEQ ID NO: 2.

136. The multispecific antibody of claim 132, wherein the ANG-2 binding moiety comprises a heavy chain variable region comprising an amino acid sequence comprising at least 85%, 90%, 95%, 96%, 97%, 98%, 99%, or 100% identity to the amino acid sequence set forth in SEQ ID NO: 11.

137. The multispecific antibody of claim 132, wherein the ANG-2 binding moiety comprises a heavy chain constant region comprising an amino acid sequence comprising at least 85%, 90%, 95%, 96%, 97%, 98%, 99%, or 100% identity to the amino acid sequence set forth in SEQ ID NO: 12.

138. The multispecific antibody of claim 132, further comprising a surrogate light chain, and wherein the surrogate light chain comprises:
a. a light chain complementarity determining region 1 (LCDR1) comprising the amino acid sequence set forth in SEQ ID NO: 23 (NDHDIGVYS);
b. a light chain complementarity determining region 2 (LCDR2) comprising the amino acid sequence set forth in SEQ ID NO: 24 (YFSQSDK); and
c. a light chain complementarity determining region 3 (LCDR3) comprising the amino acid sequence set forth in SEQ ID NO: 25 (AMGARSSVTH).

139. A nucleic acid or plurality of nucleic acids encoding the multispecific antibody of claim 132.

140. A pharmaceutical composition comprising the multispecific antibody of claim 132 and a pharmaceutically acceptable carrier, excipient, or diluent.

141. A method of treating a disease of an eye in an individual comprising administering to the individual the multispecific antibody of claim 132, thereby treating the disease of the eye.
